(19) United States
(12) Patent Application Publication
(10) Pub. No.: US 2012/0238532 A1
(43) Pub. Date: Sep. 20, 2012

(54) TREATMENT OF HUMAN OSTEOSARCOMA

(75) Inventors: Dhong Hyun Lee, Los Angeles, CA (US); Nils H. Thoennissen, Los Angeles, CA (US); H. Phillip Koeffler, Los Angeles, CA (US)

(73) Assignee: CEDARS-SINAI MEDICAL CENTER, Los Angeles, CA (US)

(21) Appl. No.: 13/512,855

(22) PCT Filed: Nov. 30, 2010
(86) PCT No.: PCT/US10/58346

§ 371 Date: May 30, 2012

Related U.S. Application Data
(60) Provisional application No. 61/265,287, filed on Nov. 30, 2009.
(60) Provisional application No. 61/314,076, filed on Mar. 15, 2010.

(51) Int. Cl. A61K 31/575; A61P 35/04; A61P 35/00

(52) U.S. Cl. 514/171

(57) ABSTRACT

The present invention describes the combination of cucurbitacin (CuB) with methotrexate (MTX) for the treatment of cancers, including osteosarcoma. It was discovered that CuB and MTX have synergistic activity against osteosarcoma, which reduces toxicities associated with both chemotherapeutic agents. The present invention also describes the use of CuB for the treatment of osteosarcoma.

### CROSS-REFERENCE TO RELATED APPLICATIONS

The present application claims the benefit of the filing date of U.S. Provisional Application No. 61/265,287 filed Nov. 30, 2009 and U.S. Provisional Application No. 61/314,076, filed Mar. 15, 2010, the disclosures of which are incorporated herein by reference in its entirety.

### FIELD OF INVENTION

This invention relates to the treatment of cancer, and in particular, the treatment of osteosarcoma.

### BACKGROUND

mTOR (mammalian target of rapamycin) is a serine-threonine kinase which serves as an integration center of many signaling pathways. By combining all intracellular and extracellular signals, mTOR regulates cell metabolism, growth, proliferation and survival. Upregulation of mTOR and related proteins are often observed in many types of human cancers (1, 2), which makes mTOR inhibitors very attractive chemotherapeutic agents.

Upregulation of mTOR occurs in human osteosarcomas (OS) and is associated with poor prognosis for these patients (3, 4). Dysregulation of upstream signaling proteins contributes to this upregulation of mTOR. According to the COSMIC library (Catalogue of Somatic Mutations in Cancer, version 49), OS shows frequent somatic mutations of RB1, TP53, BRAF, and EGFR, all of which converge to increase phosphorylation of mTOR. Increased protein expression of extracellular signal-regulated kinase (ERK) in OS can also result in enhanced mTOR signaling (5-9). Taken together, targeting mTOR may be a good therapeutic strategy for the treatment of OS.

Cucurbitacins are a group of plant-derived tetracyclic triterpenoids originally found in the plant family of Cucurbitaceae. Plants containing cucurbitacins have been known for their anti-pyretic, analgesic, anti-inflammatory, anti-microbial, and anti-tumor activities in folk medicine. They show strong antiproliferative activity against many human cancer cells as the inhibitor of the Janus kinase (JAK)-signal transducer and activator of transcription (STAT) pathway (10, 11). However, cucurbitacins can selectively inhibit different signaling pathways depending on the cancer cell type albeit the mechanisms are usually unknown (10, 11).

### SUMMARY OF THE INVENTION

In one embodiment, the invention provides a method of treating cancer in a mammal, comprising administering a quantity of cucurbitacin (CuB) with a quantity of methotrexate (MTX) to a mammal in need thereof, in an amount effective to treat the cancer. CuB and/or MTX may be a pharmaceutical equivalent, analog, derivative, salt or prodrug thereof. In one embodiment, the cancer is osteosarcoma. The administration of the combination of CuB and MTX has a synergistic effect in the treatment of cancer. The quantity of CuB and/or MTX may be administered in a low dose. Low dose administration of CuB may occur every one to three days. More prefereably, low dose administration of CuB may occur every two days. Low dose administration of MTX may occur once every one to three weeks. More preferably, low dose administration of MTX may occur every two weeks.

In a further embodiment, the invention provides a method of inducing apoptosis in cancer cells in a mammal, comprising administering a quantity of CuB with a quantity of MTX to a mammal in need thereof, in an amount effective to induce apoptosis of the cancer cells in the mammal CuB and/or MTX may be a pharmaceutical equivalent, analog, derivative, salt or prodrug thereof. In one embodiment, the cancer is osteosarcoma. The administration of the combination of CuB and MTX has a synergistic effect in the treatment of cancer. The quantity of CuB and/or MTX may be administered in a low dose. Low dose administration of CuB may occur every one to three days. More prefereably, low dose administration of CuB may occur every two days. Low dose administration of MTX may occur once every one to three weeks. More preferably, low dose administration of MTX may occur every two weeks.

In a related embodiment, the invention provides a method of preventing metastases of cancer in a mammal, comprising administering a quantity of CuB with a quantity of MTX to a mammal in need thereof, in an amount effective to prevent metastases of the cancer in the mammal CuB and/or MTX may be a pharmaceutical equivalent, analog, derivative, salt or prodrug thereof. In one embodiment, the cancer is osteosarcoma. The administration of the combination of CuB and MTX has a synergistic effect in the treatment of cancer. The quantity of CuB and/or MTX may be administered in a low dose. Low dose administration of CuB may occur every one to three days. More prefereably, low dose administration of CuB may occur every two days. Low dose administration of MTX may occur once every one to three weeks. More preferably, low dose administration of MTX may occur every two weeks.

In a related embodiment, the invention provides a method of reducing the likelihood of metastases of cancer in a mammal, comprising administering a quantity of CuB with a quantity of MTX to a mammal in need thereof, in an amount effective to prevent metastases of the cancer in the mammal. CuB and/or MTX may be a pharmaceutical equivalent, analog, derivative, salt or prodrug thereof. In one embodiment, the cancer is osteosarcoma. The administration of the combination of CuB and MTX has a synergistic effect in the treatment of cancer. The quantity of CuB and/or MTX may be administered in a low dose. Low dose administration of CuB may occur every one to three days. More prefereably, low dose administration of CuB may occur every two days. Low dose administration of MTX may occur once every one to three weeks. More preferably, low dose administration of MTX may occur every two weeks.

In another embodiment, the invention provides a composition comprising a quantity of CuB with a quantity of MTX. The composition may further comprise a pharmaceutically acceptable carrier. The composition may comprise a pharmaceutical equivalent, analog, derivative, salt or prodrug of any of CuB and/or MTX.

In another embodiment, the invention provides a kit for the treatment of cancer comprising composition comprising a quantity of CuB with a quantity of MTX and instructions for use. The composition may comprise a pharmaceutically acceptable carrier. The kit may comprise a pharmaceutical equivalent, analog, derivative, salt or prodrug of any of CuB and/or MTX.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1. Growth inhibition of human osteosarcoma (OS) cell lines by cucurbitacin B. (A) Chemical structure of cucurbitacin B. (B) Graphical representation of dose-dependent antiproliferative activity of CuB against 7 human OS cell lines (U2OS, G292, MG-63, HT-161, HOS, SAOS-2, and SJSA). Activity was measured by MTT assay after 48 hours of exposure. Measurements repeated in triplicates. Data represent the mean±standard deviation (SD; error bars).

FIG. 2. (A) Graphical representation of the effect of CuB on MG-63 and SAOS-2 cells. Cells were exposed to CuB at their ED₅₀ (70 nM for MG-63 and 50 nM for SAOS-2 cells). Time-dependent antiproliferative activity of CuB on MG-63 and SAOS-2 cells measured by pulse-exposure experiments. Cells were exposed to CuB for 2, 9, or 20 hours, washed extensively, and cultured in the absence of CuB for an additional 24, 48, and 72 hours. Cell growth was measured by MTT assay. Measurements were repeated in triplicates. Data represent mean±SD. Asterisks (**) represent p<0.001 vs DMSO control by t-test. (B) Morphological changes of MG-63 and SAOS-2 cells after exposure to cucurbitacin B. Cells with normal morphology (top) were compared to rounded cells (middle) and multinucleated cells (bottom). Rounded cells were observed after 2 hour exposure to cucurbitacin B. Multinucleated cells were observed at day 10 after a 9 hour pulse-exposure to cucurbitacin B. Cells were visualized with crystal violet staining. Representative cells are shown. 400×; scale bar=50 μm. (C) Graphical representation of G2/M cell cycle arrest after 20 hours of exposure to cucurbitacin B. Cells were stained with propidium iodide (PI) and analyzed by FACS. (D) Graphical representation of apoptosis after 72 hours of exposure to cucurbitacin B. Cells were stained with PI and Annexin V-FITC, and analyzed by FACS.

FIG. 3. Western blots showing the effect of CuB on the mTOR signaling pathway in MG-63 and SAOS-2 cells. (A) DARTS with CuB using MG-63 whole-cell lysates. MG-63 Lysates were treated with DMSO control or CuB (10, 100, or 1000 nM) at room temperature for 30 min. Samples then underwent thermolysin proteolysis followed by Western blot analysis. (B) Western blots of CuB on the phosphorylation status of mTOR and its downstream targets, S6K and 4EBP1. Cells were exposed to CuB for 2, 9, or 20 hours and analyzed by Western blotting. Same lysates were used throughout Western blot analysis. (C) Western blots of the effect of CuB on key regulators of mTOR. (D) Western blots of the effect of CuB on cell cycle-related proteins (cyclin A1, cyclin D1, and p21^{WAF}) and apoptosis-related protein (PARP). All Western blots were repeated three times for validation of the results. GAPDH protein was used as an internal loading control. CuB=cucurbitacin B. ND=not detected.

FIG. 4. (A) Graphical representation of the effect of the combination of CuB and MTX on the growth of MG-63 and SAOS-2 cells in vitro. Cells were grown in various concentrations of CuB and MTX, and their viability was determined after 48 hours by MTT assay. Numbers on the x-axis indicate the concentration (nM) of CuB and/or MTX. Samples were measured in triplicates. Data represent mean±standard deviation (SD, error bars). (B) Graphical representation of normalized isobolograms of CuB and MTX in MG-63 and SAOS-2 cells. Each point represents different concentration ratios of CuB and MTX (Table 2). Points under the line represent synergism with combination index less than 0.9. Isobolograms were generated using data in panel (A) by CalcuSyn 2.0 software. (C) The effect of CuB (70 nM) and/or MTX (50 nM) on cell cycle of MG63 cells at 12 hours of exposure. (D) Annexin V-FITC apoptosis assays of MG-63 after 72 hours of exposure to either CuB (70 nM), MTX (50 nM), or both.

FIG. 5. (A) Graphical representation of the effect of combination of CuB and MTX on the growth of MG-63 xenografts in athymic nude mice. PBS, diluent control; LD-CuB, low-dose CuB (0.5 mg/kg body weight); HD-CuB, high-dose CuB (1.0 mg/kg); LD-MTX, low-dose methotrexate (150 mg/kg); VLD-MTX, very low-dose methotrexate (50 mg/kg). Volumetric growth of MG-63 xenografts in LD-CuB groups (left) and HD-CuB groups (right). Data show mean tumor volume±standard deviation (SD, error bars) of five mice per group. Asterisks (**) represent p<0.001 vs LD-CuB or vs LD-MTX by t-test. (B) Graphical representation of body weight change of mice over the course of treatment. Data show mean body weight±SD of five mice per group. All measurements were repeated in triplicates to ensure accuracy. Asterisks (*) represent p<0.05 vs groups without LD-MTX treatment by t-test. (C) Comparison of size (top) and weight (bottom) of tumors from each group. At day 35, mice were sacrificed and tumors were excised, weighed, and fixed in 10% PBS-buffered formalin. Data represent mean volume±SD of ten tumors from five mice per group. (D) Western blot results using snap-frozen tumors from each group. Results were repeated in triplicates. GAPDH was used as a loading control.

FIG. 6. (A) Immunohistochemistry results of xenografts at the end of study (day 35). HE staining results. 100×, scale bar=250 μm. (B) Graphical representation of Ki-67 proliferation staining results. (C) Graphical representation of TUNEL apoptosis staining results. Ki-67 and TUNEL staining pictures are available in Supplementary FIGS. S1A and S1B, respectively. PBS, diluant control; LD-CuB, low-dose CuB (0.5 mg/kg body weight); LD-MTX, low-dose methotrexate (150 mg/kg); VLD-MTX, very low-dose methotrexate (50 mg/kg). Data represent mean percent positive cells±standard deviation (SD). NS=not significant. Asterisks (**) represent p<0.001 by t-test.

FIG. 7. Complete blood count (CBC). Whole-blood samples were harvested by submandibular bleeding and analyzed at the end of experiments (day 35). PBS, diluant control; LD-CuB, low-dose CuB (0.5 mg/kg body weight); LD-MTX, low-dose methotrexate (150 mg/kg); VLD-MTX, very low-dose methotrexate (50 mg/kg). Asterisks (**) represent p<0.001 by t-test. NS=not significant.

FIG. 8. (A) Ki-67 proliferation and (B) TUNEL apoptosis staining of xenografts. Positive cells (%) were counted using ImageJ and summarized in FIGS. 6B and 6C, respectively. PBS, diluant control; LD-CuB, low-dose CuB (0.5 mg/kg body weight); LD-MTX, low-dose methotrexate (150 mg/kg); VLD-MTX, very low-dose methotrexate (50 mg/kg).

### DETAILED DESCRIPTION OF THE INVENTION

The inventors' preliminary study demonstrated that CuB inhibited the growth of human OS cells, whose JAK-STAT pathway is known to be inactive (12). This led them to study what other pathways are affected by cucurbitacin B. Here, the inventors show that CuB can directly inhibit mTOR phosphorylation in human OS cells. This molecular understanding led them to explore further for possible synergism of CuB with MTX in preclinical settings.

Current chemotherapeutic regimens for OS treatment use the combination of multiple chemotherapeutic agents including HD-MTX with leucovorin rescue, doxorubicin (adriamycin), cisplatin, and ifosfamide either with or without etoposide (26). Although these regimens have remained the mainstay of OS chemotherapy for decades, none have provided any major advancement in survival compared to the original combination by Rosen et al. (27, 28). Furthermore, these regimens were only efficacious with localized OS and performed poorly with development of metastatic, recurrent OS (26). Interestingly, none of these drugs target any specific signaling pathway.

Targeting the mTOR pathway can be a good therapeutic strategy in OS because many known changes in OS converge to dysregulate the mTOR pathway. Several mTOR inhibitors in clinical trials for OS treatment support this idea. These include rapamycin (Sirolimus) with cyclophosphamide (NCT00743509) and Ridaforolimus (AP23573) as a single agent (NCT00538239) (26). However, these first-generation mTOR inhibitors (rapamycin and its derivatives) mainly inhibit the formation of mTORC1 through their binding to FK506 binding protein 12 (FKBP12) and have little or unknown effect on mTORC2.

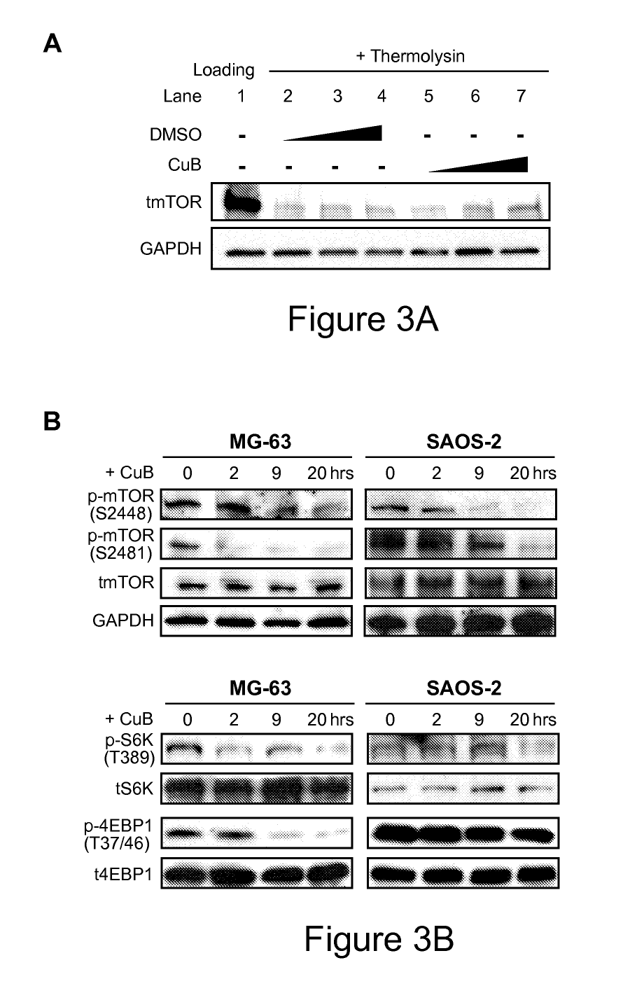

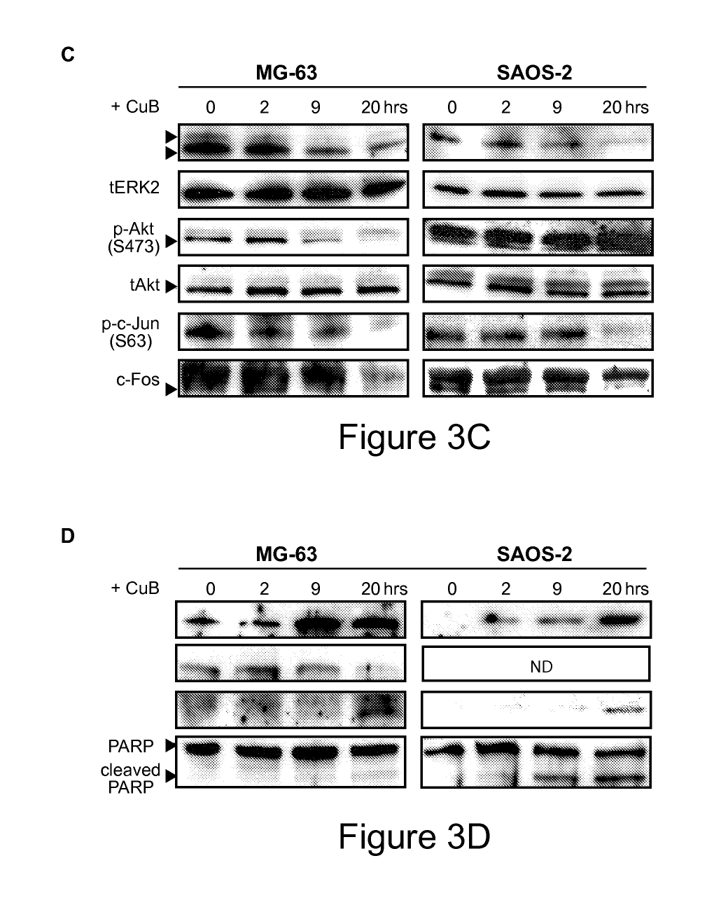

As an mTOR inhibitor, the molecular mechanism of CuB resembles those of second generation mTOR inhibitors such as Torin 1 and TORKinibs (PP242 and PP30) which directly inhibit the kinase domain of mTOR (29, 30). The phosphorylation sites of mTOR inhibited by cucurbitacin B, S2448 and S2481, are known to regulate the activity of both mTORC1 and mTORC2 (FIG. 3B) (31). Therefore, CuB can inhibit the activity of both mTOR complexes.

Initially, the inventors study was confounded by the fact that Akt and ERK, two main upstream regulators of mTOR (21), were also inhibited by CuB in OS cells Inhibition of Akt phosphorylation at S473 in MG-63 cells could be explained as feedback regulation by mTORC2 (32). Inhibition of ERK, however, raised a question whether the inhibition of mTOR is an outcome of direct inhibition by CuB or simply the indirect outcome of ERK inhibition. To answer this question, the inventors performed DARTS analysis which can help identify the interaction of drugs and their target molecules (14). Whereas the inventors observed the dose-dependent protection of mTOR protein by CuB (FIG. 3A), the inventors could not find any evidence that the ERK protein was protected by CuB (data not shown). Because the mTOR, MAPK/ERK, and Akt pathways form a complex regulatory network with each other, more studies are necessary to identify the true molecular outcome of mTOR inhibition by cucurbitacin B.

The observation of a strong synergism of CuB with MTX to suppress the growth of OS is consistent with the concept that CuB inhibits mTOR. MG-63 cells are known to be MTX resistant due to its high expression level of DHFR (33). Therefore, inhibition of mTOR by CuB can sensitize MG-63 cells to MTX by blocking RB1 phosphorylation and by decreasing cyclin D1 stability (34). Interestingly, SAOS-2 cells which lack RB and cyclin D1 (35) still showed the synergism of CuB and MTX. SAOS-2 cells are known to achieve their MTX-resistance by high expression of reduced folate carrier (RFC) rather than by high expression of DHFR (36). Therefore, synergism of CuB and MTX in SAOS-2 cells seem to follow a different mechanism although the relationship of RFC and mTOR remains unclear.

MTX is one of the essential chemotherapeutic agents for OS treatment. Nearly all successful chemotherapeutic regimens for OS include HD-MTX. However, HD-MTX is associated with some confounding issues such as appropriate administration and monitoring of the drug associated with inter- and intra-patient variability (26). Furthermore, the administration of leucovorin is almost always necessary due to severe toxicity of HD-MTX. Combined use of CuB and MTX may help to lower MTX dose as well as the need for leucovorin by reducing toxicity.

The in vivo xenograft studies successfully demonstrated that marked growth inhibition of OS cells was achievable by the combined use of LD-CuB with LD-MTX. Many xenograft studies using murine models have shown that LD-MTX alone showed poor growth inhibition (<20%) of human OS cells (24, 37). Therefore, the growth inhibition of LD-MTX combined with LD-CuB was remarkable. However, as previously reported in the xenograft studies by others, systemic toxicity by LD-MTX can still occur in this combination (23-25). This toxicity problem was resolved by lowering the dose of MTX by two thirds (VLD-MTX, 50 mg/kg body weight) without compromising the growth inhibition of OS cells.

Based on the above studies, the inventors have discovered for the first time that cucurbitacin B is a direct inhibitor of mTOR phosphorylation in human OS cells. Furthermore, they have discovered that curcubitacin B as a single agent or in combination with MTX showed promising antiproliferative activity in human OS cells. Considering that current mTORC1-specific inhibitors as a single agent are not clinically overly potent (29, 38), cucurbitacin B which can inhibit mTOR and ERK at the same time can lead to more efficient growth inhibition of OS cells. In addition, synergism of cucurbitacin B and MTX may lower the need for the currently used, highly toxic HD-MTX. The inventors' research lays the foundation for more effective therapy potentially for OS.

One embodiment of the present invention provides a method of treating cancer in a mammal in need thereof, comprising administering a quantity of MTX with a quantity of CuB to the mammal in need thereof to treat the cancer. A quantity of a pharmaceutical equivalent, analog, derivative, salt or prodrug of any of CuB and/or MTX may also be used in the method. In one embodiment, the cancer is osteosarcoma.

One embodiment of the present invention provides a method of inducing apoptosis in cancer cells in a mammal in need thereof, comprising administering a quantity of MTX with a quantity of CuB to the mammal in need thereof to induce apoptosis of the cancer cells in the mammal A quantity of a pharmaceutical equivalent, analog, derivative, salt or prodrug of any of CuB and/or MTX may also be used in the method. In one embodiment, the cancer is osteosarcoma.

One embodiment of the present invention provides a method of preventing metastases or reducing the likelihood of metastases of cancer in a mammal, comprising administering a quantity of MTX with a quantity of CuB to the mammal in need thereof to prevent the metastases of the cancer in the mammal A quantity of a pharmaceutical equivalent, analog, derivative, salt or prodrug of any of CuB and/or MTX may also be used in the method. In one embodiment, the cancer is osteosarcoma. In one embodiment, the cancer is osteosarcoma.

In one embodiment, the quantity of CuB is provided in a low dose. Low doses of CuB may be provided in the range of 0.2 mg/kg body weight to 0.6 mg/kg body weight. In one embodiment, the low dose of CuB is 0.5 mg/kg body weight. In various embodiments, the low dose of CuB may be provided every one to three days. In one embodiment, the low dose of CuB is provided every two days. In a particular embodiment, the low dose of CuB is 0.4 mg/kg body weight, every 2 days. One of skill in the art will readily be able to convert these dosages to dosages that are effective in human subjects; for example by using the method taught by Regan-Shaw et al. (Dose translation from animal to human studies revisited. FASEB J. 2008; 22(3):659-661.)

In another embodiment the quantity of MTX is also a low dose. The low dose of MTX may be in the range of 100 mg/kg body weight to 200 mg/kg body weight. In a particular embodiment, the low dose of MTX is 150 mg/kg body weight. In another embodiment the quantity of MTX is at an even lower dose. The even lower dose of MTX may be in the range of 25 mg/kg body weight to 100 mg/kg body weight. In a particular embodiment, the even lower dose of MTX is 50 mg/kg body weight. Again, one of skill in the art will readily be able to convert these dosages to dosages that are effective in human subjects.

In one embodiment, the low dose or even lower dose of MTX is administered every one to three weeks. In one embodiment, the low dose or even lower dose of MTX is administered every two weeks. In a particular embodiment, the low dose of MTX is 150 mg/kg body weight, every 2 weeks. In another particular embodiment, the even lower dose of MTX is 50 mg/kg body weight, every 2 weeks. Again, one of skill in the art will readily be able to convert these dosages to dosages that are effective in human subjects.

In one particular embodiment, a method of treating osteosarcoma in a mammal in need thereof comprises administering 0.4 mg/kg body weight of CuB to the mammal every two days; and administering 150 mg/kg body weight MTX every two weeks to the mammal to treat the osteosarcoma. In another particular embodiment, a method of treating osteosarcoma in a mammal in need thereof comprises: administering 0.4 mg/kg body weight of CuB to the mammal every two days; and administering 50 mg/kg body weight MTX every two weeks to the mammal to treat the osteosarcoma. Again, one of skill in the art will readily be able to convert these dosages to dosages that are effective in human subjects. In one embodiment, treatment of the osteosarcoma reduces the volume of the tumor.

Another embodiment of the present invention provides for a method of treating osteosarcoma comprising providing CuB and administering the CuB to a subject in need of treatment for osteosarcoma to treat the osteosarcoma. In one embodiment, the treatment inhibits the tumor growth.

In one embodiment, the quantity of CuB is provided in a low dose. Low dose of CuB may be provided in the range of 0.2 mg/kg body weight to 0.6 mg/kg body weight. In one embodiment, the low dose of CuB is 0.4 mg/kg body weight. In various embodiments, the low dose of CuB may be provided every one to three days. In one embodiment, the low dose of CuB is provided every two days. In a particular embodiment, the low dose of CuB is 0.4 mg/kg body weight, every two days. Again, one of skill in the art will readily be able to convert these dosages to dosages that are effective in human subjects.

One skilled in the art will recognize many methods and materials similar or equivalent to those described herein, which could be used in the practice of the present invention. The present invention is in no way limited to the methods and materials described. For purposes of the present invention, the following terms are defined below.

“Cancer” and “cancerous” refer to or describe the physiological condition in mammals that is typically characterized by unregulated cell growth. Examples of cancer include, but are not limited to, osteosarcoma, breast cancer, colon cancer, lung cancer, prostate cancer, hepatocellular cancer, gastric cancer, pancreatic cancer, cervical cancer, ovarian cancer, liver cancer, and bladder cancer, cancer of the urinary tract, thyroid cancer, renal cancer, carcinoma, melanoma, head and neck cancer, and brain cancer.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“Therapeutically effective amount” as used herein refers to that amount which is capable of achieving beneficial results in a patient with cancer; in particular a patient with osteosarcoma. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or lessen the disease even if the treatment is ultimately unsuccessful. Those in need of treatment include those already with cancer as well as those prone to have cancer or those in whom cancer is to be prevented. For example, in cancer treatment, a therapeutic agent may directly decrease the pathology of cancer cells, or render the tumor cells more susceptible to treatment by other therapeutic agents or by the subject's own immune system.

In various embodiments, the CuB and/or the MTX may be provided as pharmaceutical compositions including a pharmaceutically acceptable excipient along with a therapeutically effective amount of the CuB and/or the MTX. In other embodiments, CuB and/or the MTX may be provided as pharmaceutical equivalents, analogs, derivatives, salts or prodrugs thereof. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective amount of the CuB and/or the MTX can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

The present invention is also directed to a kit to treat cancer in a mammal in need thereof; in particular, osteosarcoma. The kit is useful for practicing the inventive method of treating cancer and in particular treating osteosarcoma. The kit is an assemblage of materials or components, including at least one of the inventive compositions. Thus, in some embodiments the kit contains a composition including CuB and/or the MTX as described above. Alternatively, CuB and/or the MTX may be provided as pharmaceutical equivalents, analogs, derivatives, salts or prodrugs thereof.

The exact nature of the components configured in the inventive kit depends on its intended purpose. For example, some embodiments are configured for the purpose of treating osteosarcoma. In one embodiment, the kit is configured particularly for the purpose of treating mammalian subjects. In another embodiment, the kit is configured particularly for the purpose of treating human subjects. In another embodiment, the kit is configured for treating adolescent, child, or infant human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat osteosarcoma or to reduce the tumor size. Instructions for use may include instructions to administer a low dose of CuB every one to three days, or every two days to the mammal; instructions to administer a low dose of MTX every one to three weeks, instructions to administer an even lower dose of MTX every one to three weeks, or every two weeks to the mammal. Particularly, instructions for use may include instructions to administer 0.4 mg/kg of CuB every two days to the mammal and to administer 150 mg/kg or 50 mg/kg of MTX to the mammal every two weeks. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in chemotherapy. As used herein, the term “package” refers to a suitable solid matrix or material such as glass, plastic, paper, foil, and the like, capable of holding the individual kit components. Thus, for example, a package can be one or more glass vials used to contain suitable quantities of an inventive composition containing CuB and/or MTX. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Osteosarcoma Cell Culture

7 human OS cell lines (U2OS, G292, MG-63, HT-161, HOS, SAOS-2, and SJSA) were used in the study. Each cell line except HT-161 was obtained from the American Type Culture Collection (ATCC, Rockville, Md.). HT-161 was obtained from Dr. Emil Bogenmann (13). All cell lines were tested and authenticated prior to use according to cell line verification test recommendations by ATCC. OS cell lines were maintained in DMEM medium (Mediatech Inc., Herndon, Va.) supplemented with 10% fetal bovine serum (FBS; Atlanta Biological, Lawrenceville, Ga.) in a humidified incubator at 37° C. supplied with 5% CO₂. Only the cells in exponential growth phase were used in the study.

### Example 2

### Chemical Compounds

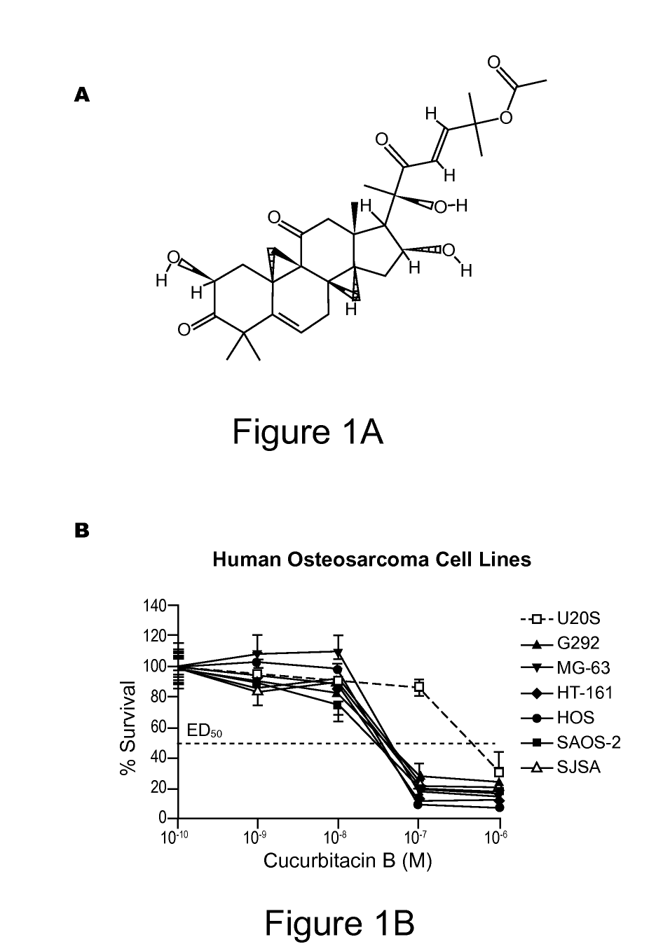

CuB (CKBP002, FIG. 1A) was generously provided by CK Life Sciences International (Holdings) Inc. (Hong Kong, China). Pure CuB crystal was solubilized with 100% ethanol to 10⁻² M and diluted with phosphate-buffered saline (PBS) to a stock concentration of 10⁻⁴ M. MTX (MTX; Affymetrix-USB, Cleveland, Ohio) was dissolved in 0.1 M sodium carbonate buffer (pH=9.6) to a stock concentration of 10⁻2 M. All chemical compounds were freshly dissolved on the day of the experiment.

### Example 3

### Measurement of Cell Growth and Survival by MTT Assay

Cell growth and survival were measured by 3-(4,5-dimethylthiazol-2-yl)-2,5-diphenyltetrazolium bromide (MTT) assay. For dose-response tests, all OS cells were seeded in 96-well plates and exposed to media with various concentrations of DMSO (control), CuB (10⁻⁶-10⁻⁹ M), or MTX (10⁻⁶-10⁻⁹ M). After 48 hours of incubation, 10 μl of 5 mg/ml MTT solution (Sigma-Aldrich, St. Louis, Mo.) was added to each well and incubated further for 2 hours. 100 μl of 20% SDS solution was added to stop the reaction and the absorbance was measured at 540 nm using ELISA reader, and ED50 was calculated. For pulse-exposure experiments, MG-63 and SAOS-2 cells were seeded in 96-well plates and exposed to CuB at their ED50. After 2, 9, and 20 hours of exposure, cells were washed twice with PBS and incubated further in CuB-free media. Cell growth was checked by MTT assay at 24, 48, and 72 hours.

### Example 4

### Cell Cycle Analysis and Apoptosis Assay

MG-63 and SAOS-2 cells were seeded in 6-well plates and exposed to either DMSO (control), CuB, or MTX at their previously calculated ED50. Cells were harvested and fixed with 70% ethanol at regular time intervals. Fixed cells were stained with propidium iodide (PI) for flow cytometry analysis using BD FACScan (BD Biosciences, San Jose, Calif.). Distribution of cell cycle was analyzed by ModFit LT V2.0 software (Verity Software House, Topsham, Me.). For apoptosis assay, MG-63 and SAOS-2 cells were seeded in 6-well plates and exposed to DMSO (negative control), 200 μM H₂O₂ (positive control), as well as either CuB or MTX at their ED50. After 24, 48, and 72 hours of exposure, cells were harvested and stained with PI and fluorescein isothiocyanate (FITC) using Annexin V-FITC apoptosis detection kit (BD Biosciences) according to the manufacturer's protocol. Cells were subjected to flow cytometry analysis within one hour of staining.

### Example 5

### Drug Affinity Responsive Target Stability (DARTS) Analysis

Direct interaction of CuB and mTOR was assessed by DARTS as described in the original article with some modifications (14). Briefly, confluent MG-63 cells were harvested and lysed with lysis buffer (1% Triton X-100, 150 mM NaCl. 100 mM Tris-HCl, pH 7.4, 1 mM EDTA, and 1 mM EGTA, pH 8.0) supplemented with protease inhibitor (Roche, San Francisco, Calif.), 0.2 mM PMSF (Roche), and 0.2 mM Na₃VO₄ (Sigma). Cell lysates were incubated with 0.1 volume of increasing concentrations (10-1000 nM) of either DMSO or CuB at room temperature for 30 min. Lysates were then proteolyzed with thermolysin (Sigma) at room temperature for 3 min. Reaction was stopped by adding 0.5 M EDTA solution and 2× Laemmli sample buffer (Bio-Rad, Hercules, Calif.) with 5% β-mercaptoethanol (Sigma). Western blotting was performed as described below.

### Example 6

### Western Blotting

Changes in protein level by CuB were checked by Western blotting. MG-63 and SAOS-2 cells were seeded in 6-cm dishes and exposed to DMSO (control), as well as either CuB or MTX at their ED50. After 2, 9, and 20 hours of exposure, cells were harvested and immediately lysed with RIPA buffer (Millipore—Upstate, Temecula, Calif.) supplemented with protease inhibitor cocktail (Roche, San Francisco, Calif.), 0.5 mM PMSF (Roche), and 50 mM NaF (Sigma). After 20 minutes of incubation on ice, protein concentrations were measured by Bradford assay with Bio Rad protein assay solution (Bio-Rad, Hercules, Calif.) and samples were adjusted to the same protein concentration. Samples were mixed with 2× Laemmli sample buffer (Bio-Rad) with 5% β-mercaptoethanol (Sigma) and incubated at 95° C. for 10 min to denature the protein. After brief incubation on ice, proteins were separated by SDS-PAGE, transferred to nitrocellulose membrane (Sigma), and blotted with antibodies from either Cell Signaling Technology (Danvers, Mass.) or Santa Cruz Biotechnology (Santa Cruz, Calif.). The list of antibodies is provided in Table 1. Target proteins were visualized by Supersignal West Dura Substrate (Thermo-Pierce, Rockford, Ill.). GAPDH protein was used as a loading control.

TABLE 1
Antibodies

| Antibody | Phosphorylation Site | Company | Catalog No |
|---|---|---|---|
| phospho-mTOR | Ser 2448 | Cell Signaling | 2971 |
| phospho-mTOR | Ser 2481 | Assay Biotech | A0688 |
| total mTOR | | Assay Biotech | B7156 |
| phospho-S6K (p70) | Thr 389 | Assay Biotech | A0533 |
| total S6K (p70) | | Cell Signaling | 9202 |
| phospho-4EBP1 | Thr 37/Thr 46 | Cell Signaling | 9459 |
| total 4EBP1 | | Cell Signaling | 9452 |
| phospho-ERK1/2 | Tyr204 | Santa Cruz | sc-7383 |
| (p44/p42) | | | |
| total ERK2 | | Santa Cruz | sc-154 |
| phospho-Akt | Ser 473 | Cell Signaling | 9271 |
| total Akt | | Cell Signaling | 9272 |
| phospho-c-Jun | Ser 63 | Santa Cruz | sc-822 |
| c-Fos | | Santa Cruz | sc-52 |
| phospho-STAT3 | Tyr705 | Cell Signaling | 9145 |
| total STAT3 | | Cell Signaling | 9132 |
| phospho-STAT5 | Tyr 694 | Cell Signaling | 9351 |
| total STAT5 | | Cell Signaling | 9363 |
| phospho-JAK2 | Tyr 1007/Tyr 1008 | Cell Signaling | 3771 |
| total JAK2 | | Cell Signaling | 3229 |

### Example 7

### Growth of MG-63 Xenografts in Athymic Nude Mice with In Vivo Treatment

All animal experiments strictly followed the guidelines of Cedars-Sinai Medical Center and the National Institute of Health (NIH). Female nu/nu athymic nude mice (5-6 weeks old; average weight 21 g; specific pathogen-free) from Harlan Laboratories (Indianapolis, Ind.) were maintained in pathogen-free condition with sterilized chow and water. 5×10⁶ cells of MG-63 cells were mixed with 200 μl of Matrigel solution (BD Biosciences) per injection, and the mixture was injected subcutaneously on the upper flanks of nude mice. After 24 hours, tumor size was measured, and any outliers were ruled out by one-way analysis of variance (ANOVA) test. 5 mice were randomly assigned to each experimental group: (1) PBS (diluent-specific control); (2) low-dose CuB (LD-CuB, 0.5 mg/kg body weight); (3) high-dose CuB (HD-CuB, 1.0 mg/kg); (4) low-dose methotrexate (LD-MTX, 150 mg/kg); (5) LD-CuB with LD-MTX; (6) HD-CuB with LD-MTX; and (7) LD-CuB with very low-dose MTX (VLD-MTX, 50 mg/kg). The dose of LD-MTX was the murine equivalent of the human dose of LD-MTX. The conversion was made using dose translation formula by Reagan-Shaw et al., (15). Intraperitoneal (i.p.) injections of PBS or CuB were administered three times a week and MTX was injected once every 2 weeks. Body weights were monitored every 2 days. Tumor size was measured every 2 days, and the tumor volume was calculated using the following formula: A (length)×B (width)×C (height)×0.5236 (16). The experiment was stopped at day 35, and all mice were sacrificed. The presence of metastatic spread was examined macroscopically at the time of autopsy followed by histological examination. At least two tumors from each group were snap-frozen in liquid nitrogen for Western blotting.

### Example 8

### Immunohistochemistry (IHC)

At autopsy, tumors and internal organs including liver, spleen, kidneys were excised, weighed, and then fixed in 10% PBS-buffered formalin and maintained in 70% ethanol. For IHC, fixed tumors and organs were embedded in paraplast (Oxford Labware, St. Louis, Mo.), cut in 6 μm thick sections, and stained with hematoxylin and eosin (HE) for histopathological examination.

For Ki-67 proliferation assay, tumor sections were deparaffinized with xylene and rehydrated through graded ethanol. Endogenous peroxidase activity was blocked with 3% hydrogen peroxide in methanol. Heat-induced antigen retrieval (HIER) was carried out in 10 mM citrate buffer (pH=6.0) using a vegetable steamer at 95° C. for 25 mM Tumor sections were incubated with a mouse monoclonal antibody for human Ki-67 (M7240, 1:100 dilution; Dako, Carpinteria, Calif.) followed by MACH2 Mouse HRP-Polymer mouse secondary antibody (MHRP520L, Biocare Medical, Concord, Calif.). After incubation, tumor sections were stained with diaminobenzidine (DAB) and counterstained with hematoxylin. Terminal deoxynucleotidyl transferase dUTP nick end labeling (TUNEL) apoptosis assay was performed using ApopTaq Plus Peroxidase In Situ Apoptosis Kit (Millipore) according to the manufacturer's protocol. Percent positive cells in representative microscopic fields were counted using ImageJ (17).

### Example 9

### In vivo Toxicity Test

Whole blood samples were obtained by submandibular bleeding and serum samples were harvested with serum separator tubes (BD Biosciences). Blood count and serum chemistry results were obtained by the Hemagen Analyst Benchtop Chemistry System (Hemagen Diagnostics, Inc. Columbia, Md.). Colony-forming cell (CFC) assays were performed as previously described (18).

### Example 10

### Statistical Analysis

All in vitro and in vivo experiments were repeated at least three times to ensure reproducibility. Two-tailed student t-test was used to compare differences between two groups. One-way ANOVA test was used to compare differences among three or more groups. P-values less than or equal to 0.05 were considered statistically significant in both tests.

Synergism between CuB and MTX in vitro was determined quantitatively by isobologram and combination index (CI) analysis adapted from the median-principle methods of Chou and Talalay (19, 20). Calcusyn 2.0 (Biosoft, Ferguson, Mo.) was used for CI analysis.

### Example 11

### Effect of CuB on Human Osteosarcoma (OS) Cell Lines In Vitro

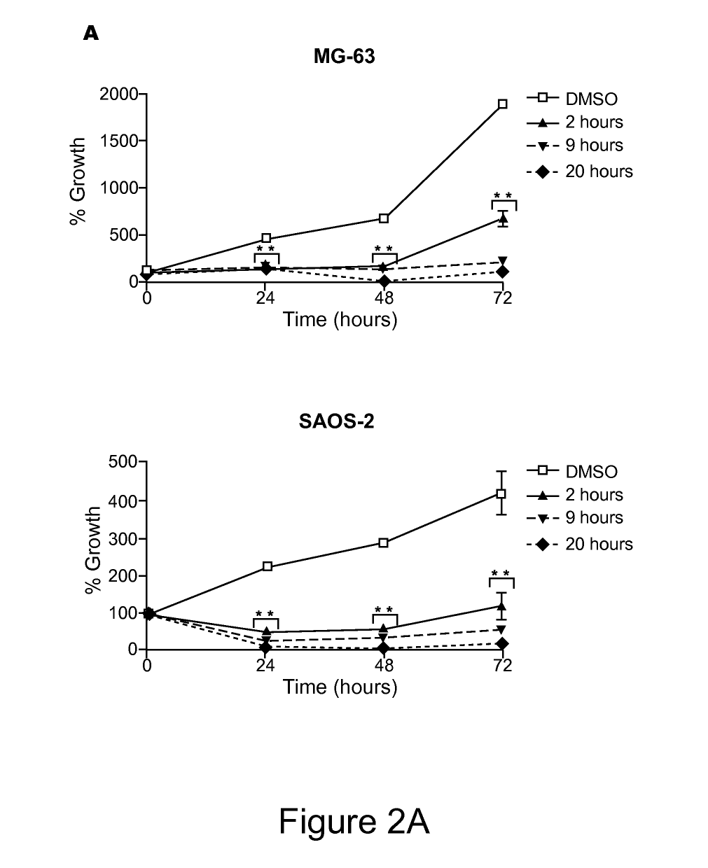

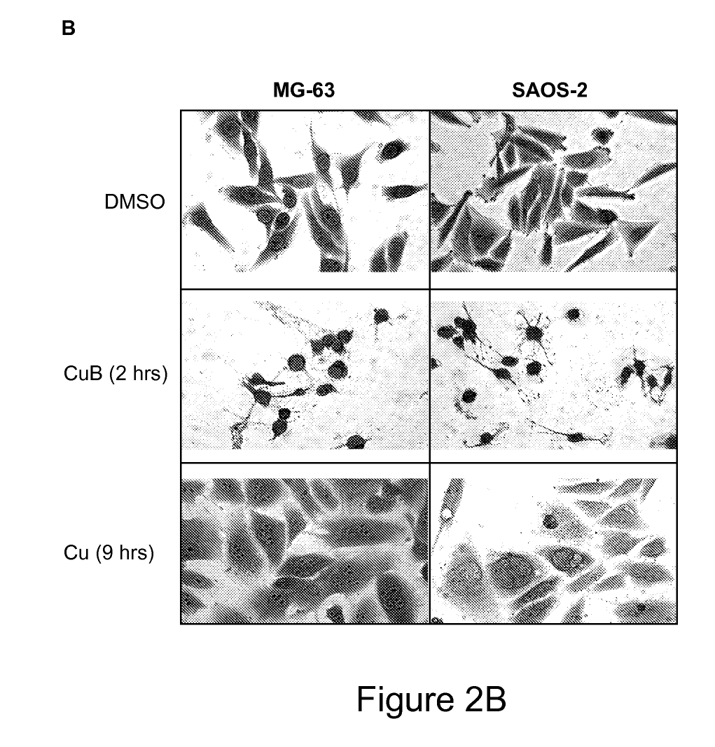

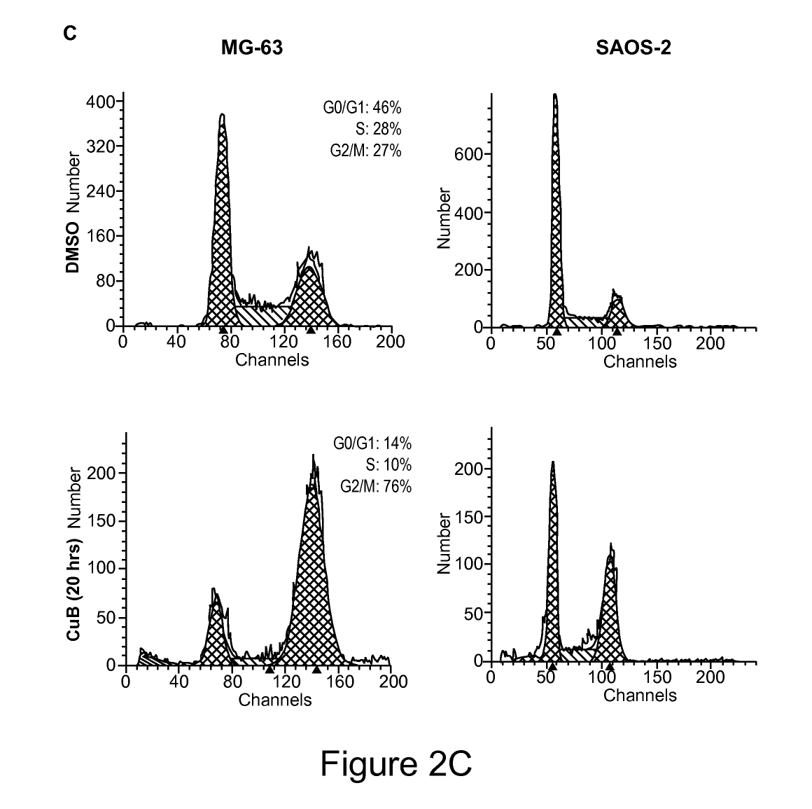

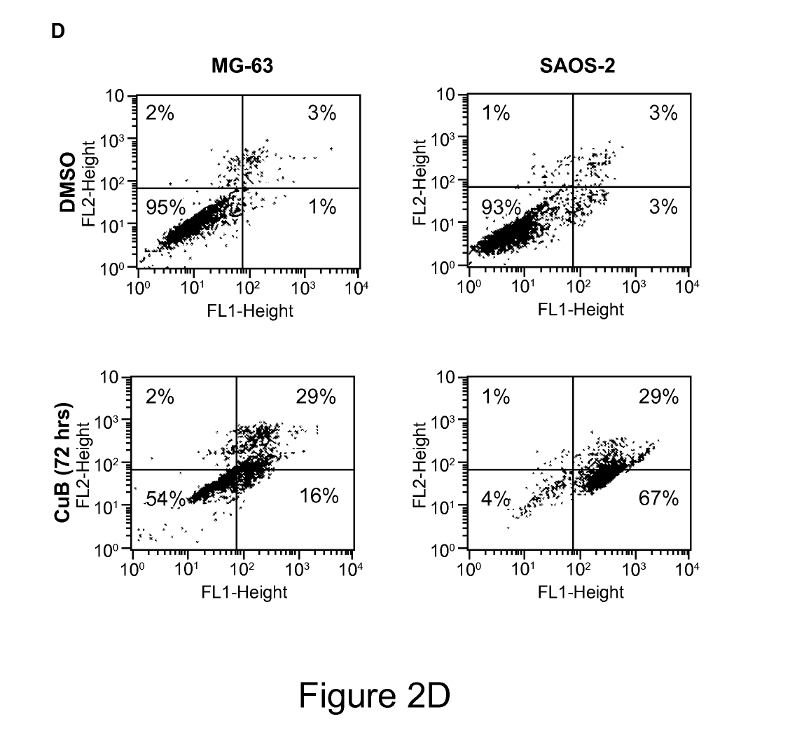

Seven human OS cell lines (U2OS, G292, MG-63, HT-161, HOS, SAOS-2, and SJSA) were exposed to increasing concentrations of cucurbitacin B, and dose-responses were determined (FIG. 1B). Cells showed similar ED₅₀ values of approximately 50 nM, except U2OS (500 nM). When MG-63 and SAOS-2 cells were pulse-exposed at their corresponding ED₅₀ (70 nM for MG-63 and 30 nM for SAOS-2 cells) for 2, 9, and 20 hours, longer exposure to CuB induced more cell death (FIG. 2A). Together, the cytotoxic activity of CuB in human OS cells was dose- and time-dependent.

Exposure to CuB induced morphological changes in MG-63 and SAOS-2 cells. Rapid loss of pseudopodia and rounding was observed in both cells after 2 hour exposure to CuB (FIG. 2B, middle panels). 9 hour-exposure to CuB resulted in multinuclearity which was not reversed by CuB removal (FIG. 2B, bottom panels).

Other responses of MG-63 and SAOS-2 cells to CuB exposure included G2/M cell cycle arrest and apoptosis. The G2/M phase increased around 3-fold for both MG-63 (27% to 76%) and SAOS-2 cells (15% to 42%) after 20 hours of exposure to CuB (FIG. 2C). Number of apoptotic cells increased to 44% in MG-63 cells and 96% in SAOS-2 cells after 72 hours of exposure (FIG. 2D).

### Example 12

### Inhibition of mTOR Phosphorylation by Cucurbitacin B

DARTS analysis demonstrated direct interaction of CuB with mTOR protein (FIG. 3A). CuB showed dose-dependent protection of mTOR protein from thermolysin proteolysis (lanes 5, 6, and 7) whereas DMSO control at the same concentrations did not (lanes 2, 3, and 4). Binding of CuB to mTOR was associated with the inhibition of mTOR phosphorylation at S2448 and S2481 without affecting the total level of mTOR in MG-63 and SAOS-2 cells (FIG. 3B). Inhibition of downstream targets of mTOR such as S6K and 4EBP1 was consistent with the inhibition of mTOR (FIG. 3B).

ERK and Akt, two main regulators of mTOR phosphorylation (21), were also affected by CuB (FIG. 3C). The compound inhibited ERK phosphorylation without changing the total levels of ERK in both cells. Decreased level of phospho-c-Jun and subsequent decrease in c-Fos expression was associated with decreased levels of ERK. Inhibition of Akt phosphorylation at S473 was observed in MG-63 cells, but not in SAOS-2 cells. Total level of Akt remained unaffected in both cells.

Consistent with cucurbitacin B-mediated G2/M arrest and apoptosis, the compound increased the protein levels of cyclin A and cyclin-dependent kinase inhibitor 1 (p21^{WAF1}) in both cells (FIG. 3D). Decrease in the levels of cyclin D1 occurred in MG-63 but not in SAOS-2 cells. The apoptosis marker protein, poly ADP-ribose polymerase (PARP), showed cleavage of the precursor molecules in both cells.

### Example 13

Synergistic Effect of CuB and MTX In Vitro

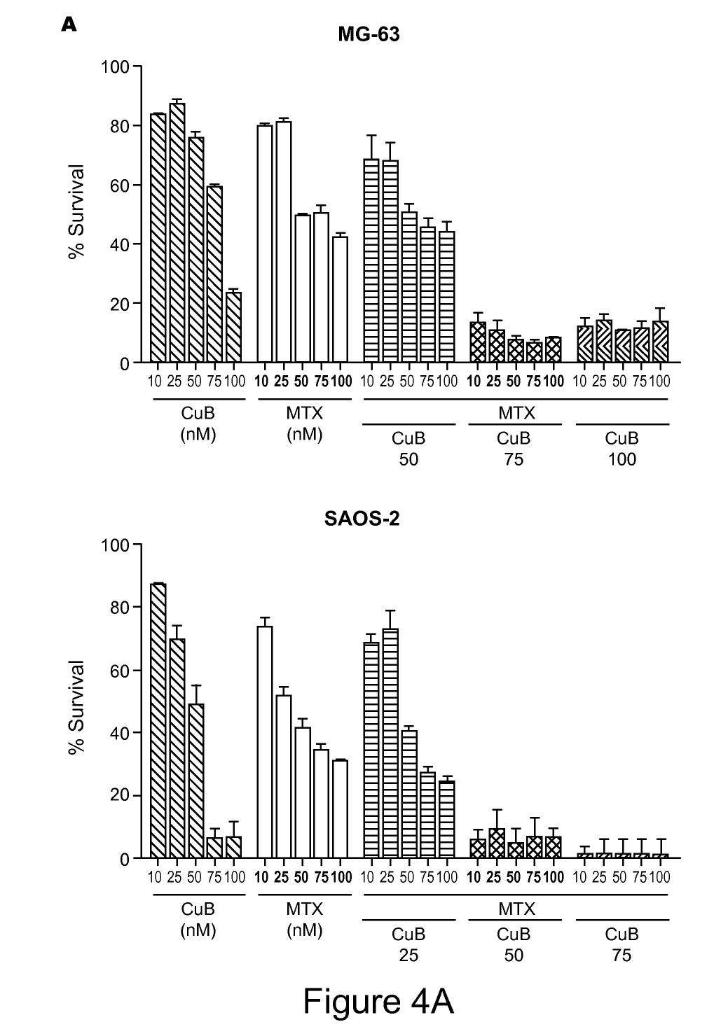

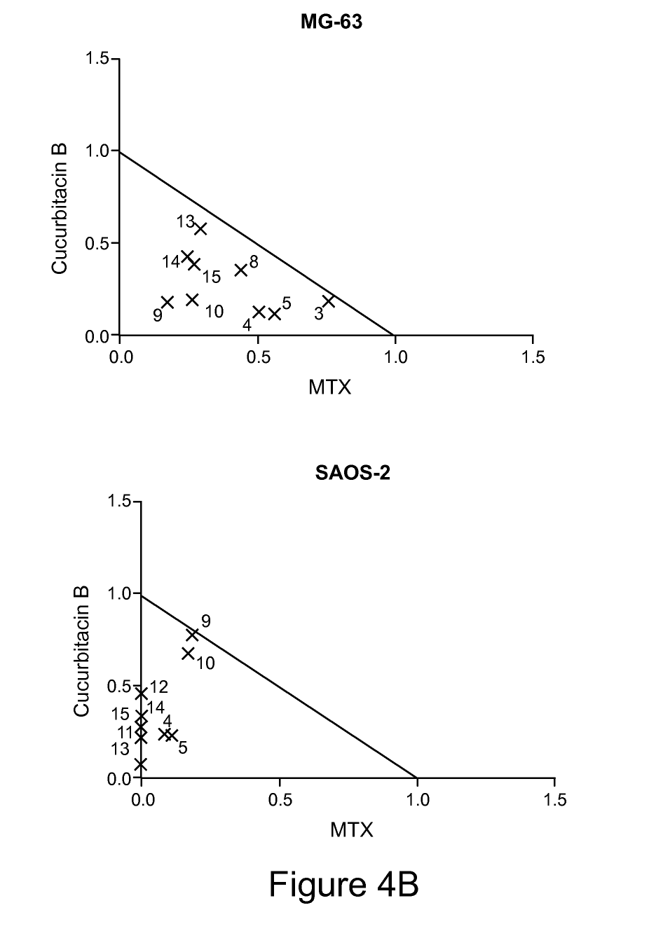

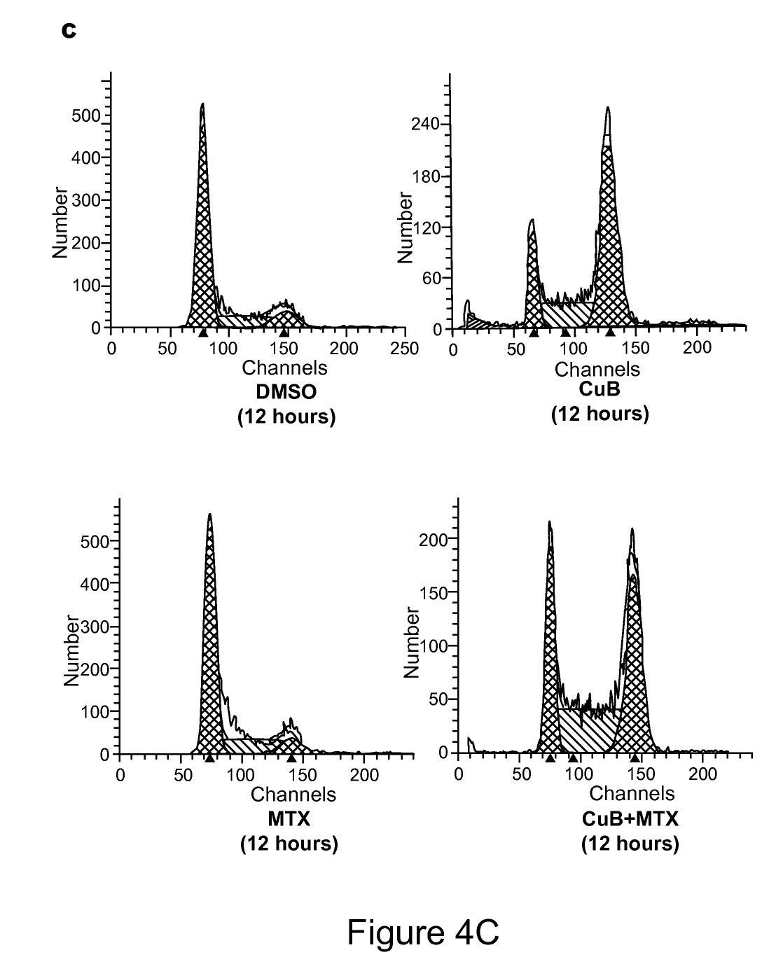

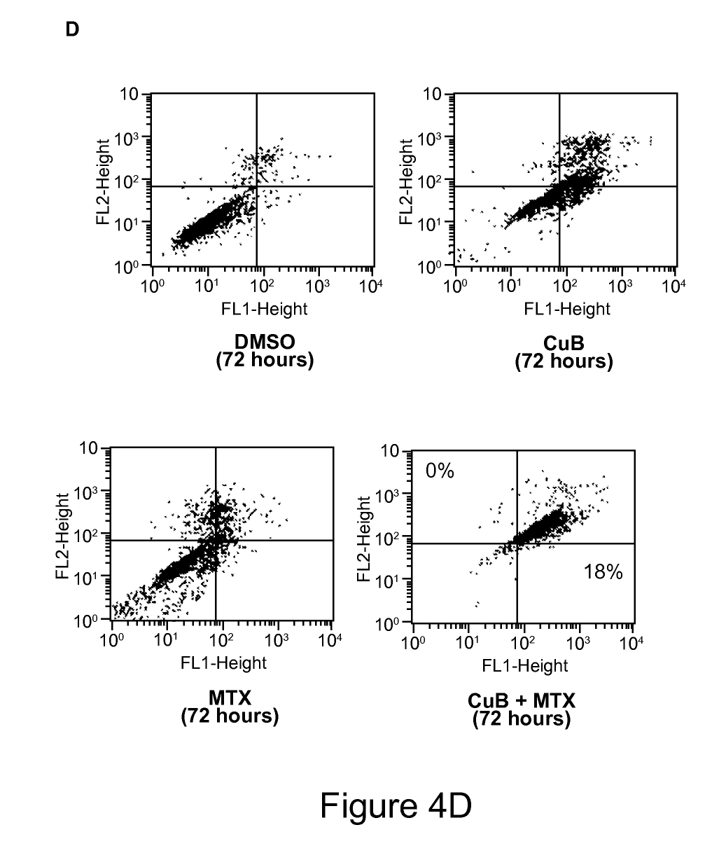

From an understanding of the molecular mechanism of action of cucurbitacin B, the inventors hypothesized that CuB might synergize with MTX. As an antimetabolite, MTX inhibits dihydrofolate reductase (DHFR) which plays a vital role in DNA synthesis. Since the expression of DHFR is known to be S-phase specific (22), the inventors hypothesized that the rapid G2/M arrest by CuB would lower the level of DHFR expression and augment the inhibitory action of MTX. To test this hypothesis, MG-63 and SAOS-2 cells were exposed to various concentrations of cucurbitacin B, MTX, or both for 72 hours and cell viability was examined. Synergism of CuB and MTX was observed at most of the concentration ratios in both cell lines (FIG. 4A). Statistical analysis using an isobologram confirmed that most concentration ratios had CI values less than 0.9 indicating synergism (FIG. 4B, Table 2).

Markedly enhanced activity of MTX combined with CuB was found when examining their effects on the cell cycle and apoptosis. CuB enhanced S-phase arrest by MTX in MG-63 cells (FIG. 4C). When MTX was used alone at its ED₅₀ (50 nM, 48 hours), the earliest sign of S-phase arrest was observed at 48 hours of exposure (data not shown). When MTX and CuB at their ED₅₀ were used in combination, increased S-phase arrest (from 27% to 38%) was observed at 12 hours of exposure. Furthermore, the combination of CuB and MTX enhanced the apoptosis of the MG-63 cells (FIG. 4D). CuB or MTX alone at their ED₅₀ caused 44% and 30% of the cells to become apoptotic after 72 hours of exposure, respectively. Together, both compounds at the same concentrations resulted in 97% apoptosis of the cells.

TABLE 2
Concentration ratios of CuB and methotrexate (MTX) used for combination index
(CI) analysis. Ratios with synergism (CI < 0.9) are shaded in gray.

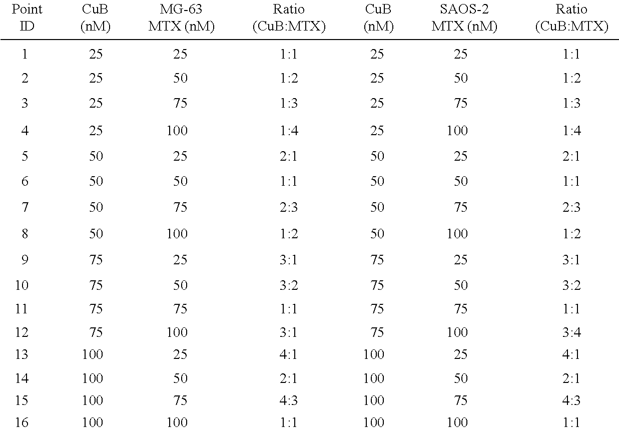

### Example 14

### Effect of CuB with MTX on Human OS Xenograft In Vivo

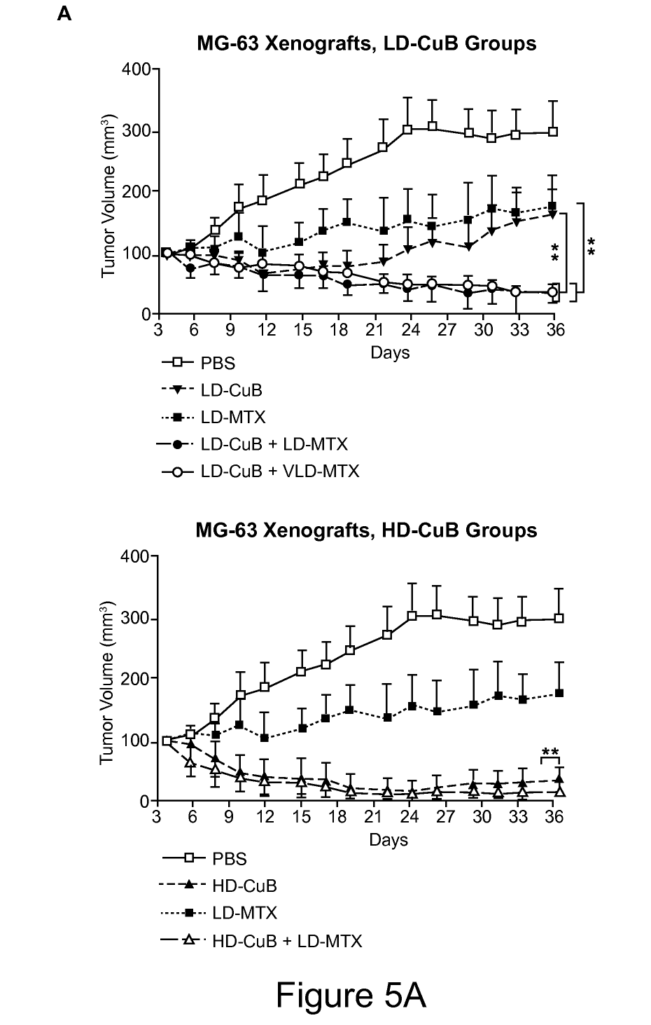

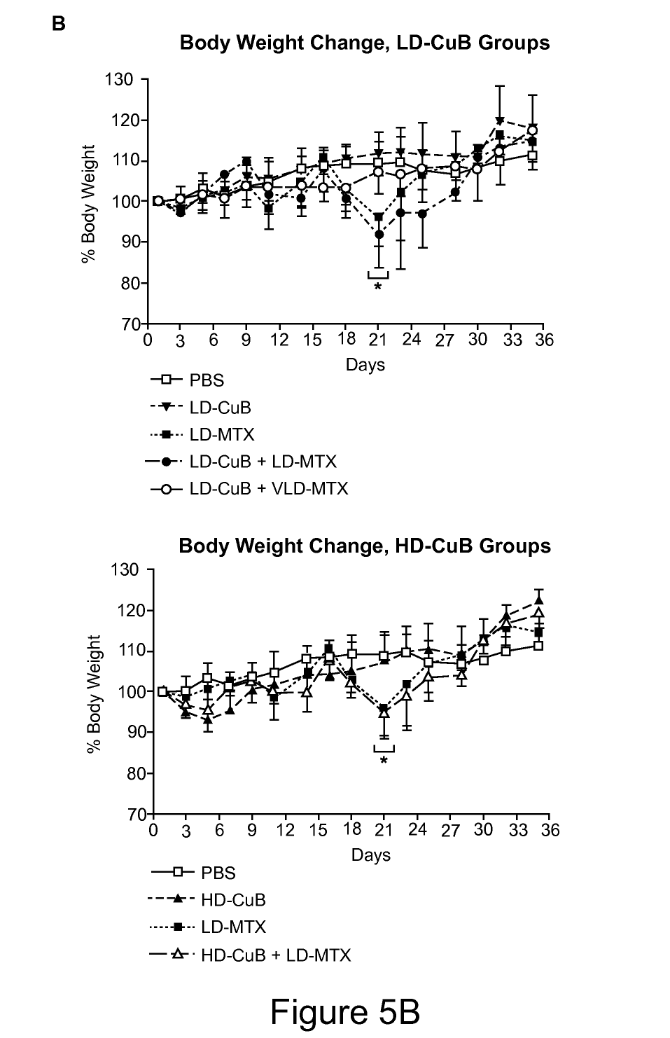

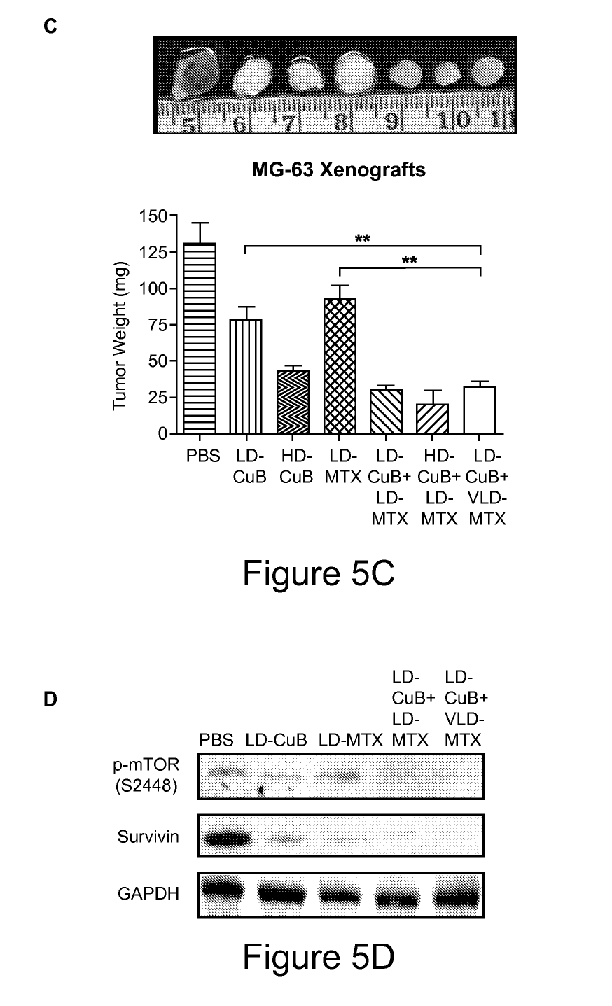

Based on the synergism of CuB and MTX in vitro, the inventors extended the experiments to the preclinical settings using a murine model. As summarized in FIG. 5A, low-dose CuB (LD-CuB, 0.5 mg/kg body weight) or low-dose MTX (LD-MTX, 150 mg/kg) as single agents allowed the tumor volume to increase slightly from its original size. In contrast, tumors were barely detectable when the two were combined at the same concentrations. Strikingly, the effect persisted even when the dose of MTX was decreased by two thirds (very low dose (VLD)-MTX, 50 mg/kg). No significant difference was found between these combination groups (LD-CuB+LD-MTX and LD-CuB+VLD+MTX) (p=0.38). At day 35, the average volumetric decrease of tumor in both combination groups was 79% vs LD-CuB group (p<0.001) and 80% vs LD-MTX group (p<0.001) (FIG. 5A, left). CuB at a high dose (HD-CuB, 1.0 mg/kg) either alone or in combination with LD-MTX showed a strong growth inhibition (FIG. 5A, right). However, no synergism was observed (p=0.71).

During treatment, minor signs of toxicity were observed in all of the treatment groups except those who received LD-MTX, either as a single agent (LD-MTX) or in combination with CuB (LD-CuB+LD-MTX and HD-CuB+LD-MTX). Some mice in these groups developed a 10% body weight loss (p<0.05) at 48 hours after MTX injection (FIG. 5B). Other side-effects included sluggish movements, occasional diarrhea, and a patchy skin rash in 60% of these mice. These side-effects by MTX have been previously reported by other groups (23-25). Strikingly, no changes in body weight were observed when the MTX dose was lowered by two thirds (LD-CuB+VLD-MTX). Two other groups (LD-CuB and HD-CuB) had an initial weight loss of up to 5% in week 1, but weights returned to normal in subsequent weeks (FIG. 5B). No other side-effects were observed.

Decrease in tumor volume was also verified by their decrease in tumor weights (FIG. 5C). At day 35, the decrease in average tumor weight of LD-CuB+LD-MTX group was 62% vs LD-CuB group (p<0.001) and 81% vs LD-MTX group (p<0.001). Likewise, the LD-CuB+VLD-MTX group showed a similar decrease in average tumor weight (69% vs LD-CuB group and 85% vs LD-MTX group, p<0.001). Western blots from the snap-frozen tumors showed decreased amount of phospho-mTOR (S2448) and its downstream target survivin in those mice treated with LD-CuB (FIG. 5D).

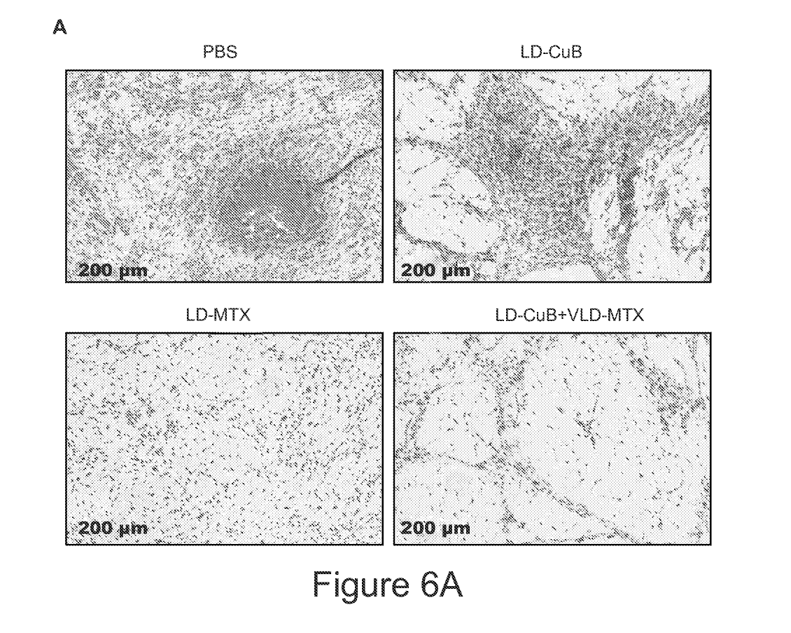

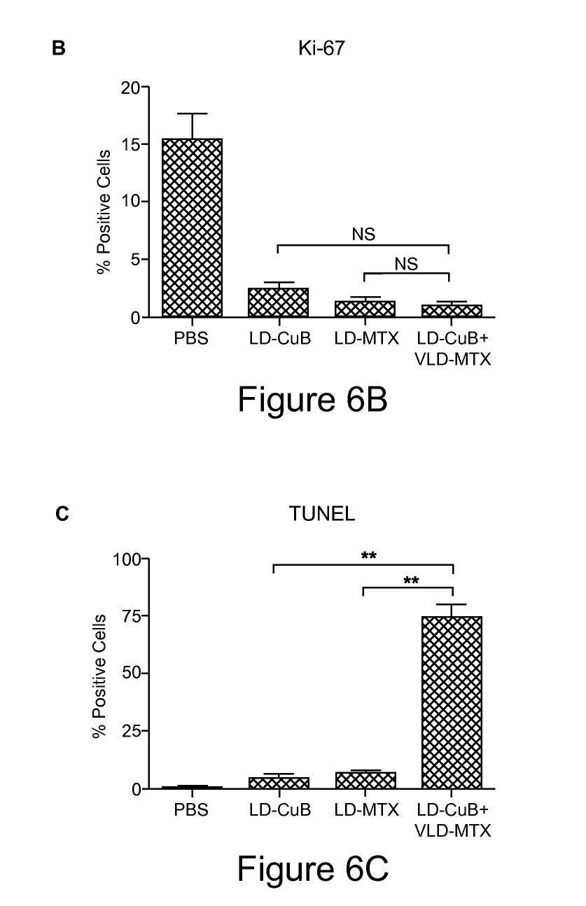

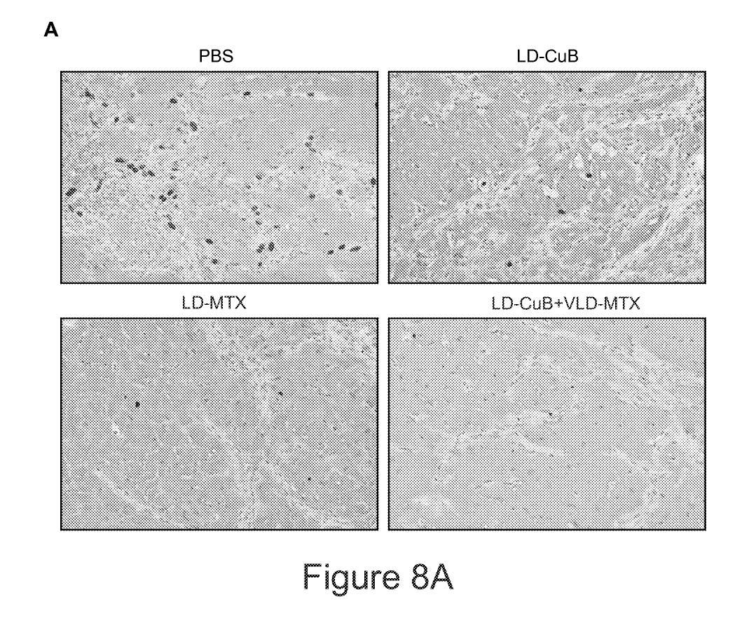

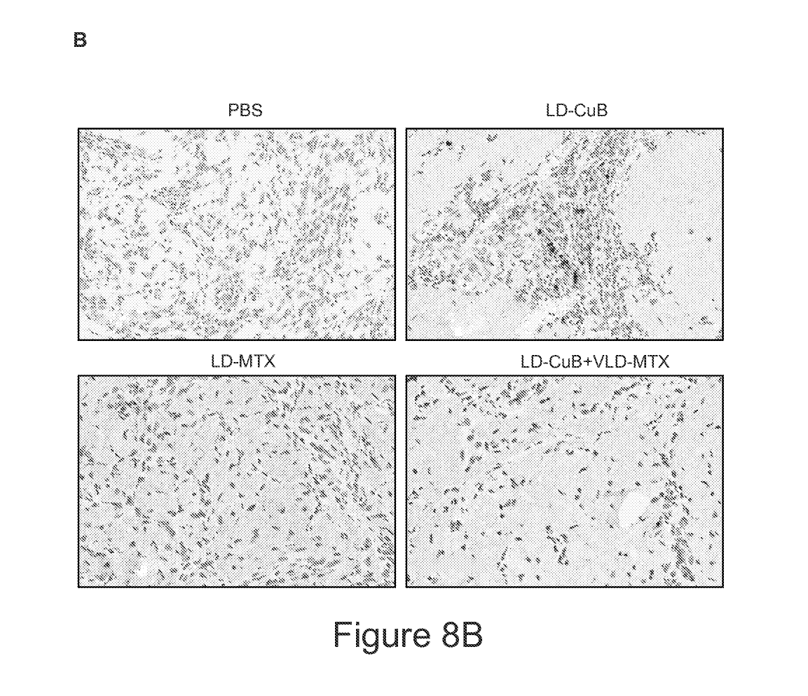

IHC further confirmed the inhibition of tumor growth. Whereas HE-stained tumors in the PBS control group showed high tumor cell density and numerous blood vessels, all treatment groups showed decreased tumor area and smaller blood vessels (LD-CuB), less number of blood vessels (LD-MTX), or both (LD-CuB+VLD-MTX) (FIG. 6A). Ki-67 proliferation staining showed marked decrease in Ki-67 positive cells in all treatment groups (FIGS. 6B and 8A). However, no significant difference was found among the treatment groups (p=0.34). On the contrary, apoptosis as measured by TUNEL-positive cells was markedly elevated in the combination group compared to the single agent groups (FIGS. 6C, and 8B).

### Example 15

### Toxicity Study of CuB and/or MTX In Vivo

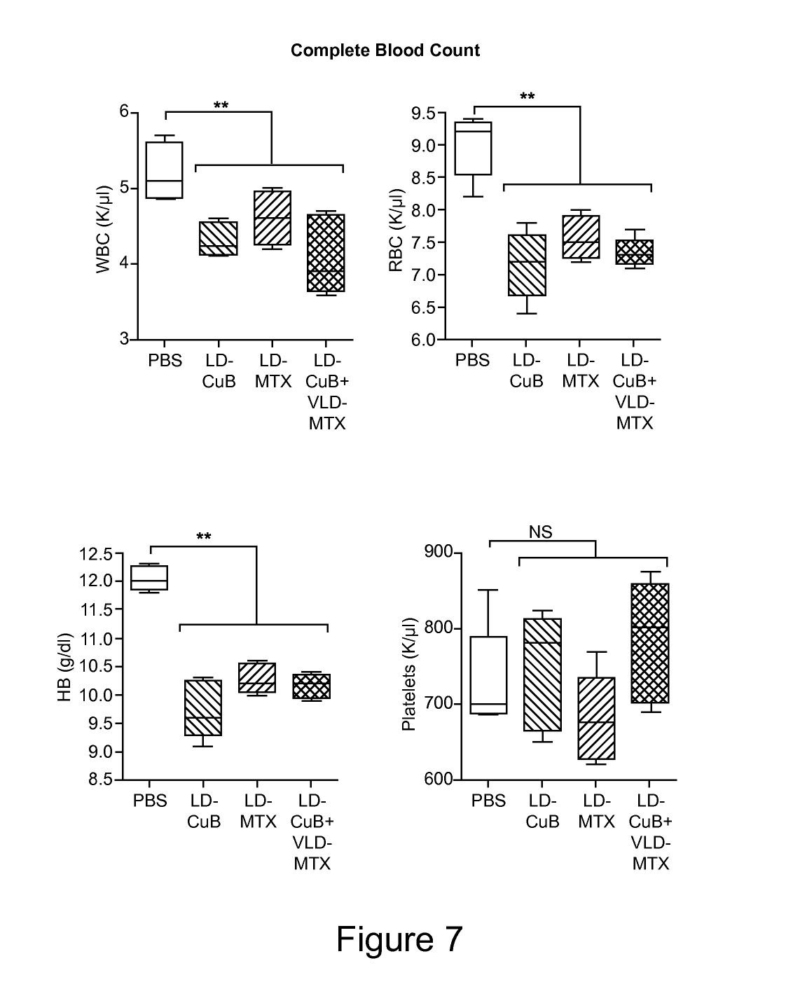

At autopsy, no signs of metastasis were found in any of the treatment groups whereas the control group had metastatic spread of tumors in various organs and regions such as mediastinum and periosteum (data not shown). Major organs such as spleen, liver, and kidney did not show any significant changes in their weight (p=0.25) in all treatment groups (Table 3). No sign of organ damage was morphologically observed (data not shown). Blood test results showed that most treatment groups had a reduction of their red blood cells (RBC), white blood cells (WBC), and hemoglobin (Hb) (p<0.001), but not platelets (p=0.15) (FIG. 7, Table 4). Serum chemistry studies did not show any significant changes between control and treatment groups (Table 5). Bone marrow clonogenic assays showed decreased number of CFU-GEMM, CFU-GM, and BFU-E hematopoietic progenitor cells in all treatment groups (p<0.001) (Table 6).

TABLE 3
Effect of CuB and/or MTX on organ weight in mice

| Treatment group | Spleen (g) | Liver (g) | Kidney (g) |
|---|---|---|---|
| Untreated | 140 ± 1 | 1200 ± 113 | 335 ± 1 |
| LD-CuB¹ | 117 ± 6 | 1247 ± 60 | 360 ± 1 |
| HD-CuB² | 110 ± 1 | 1260 ± 141 | 370 ± 1 |
| LD-MTX³ | 135 ± 7 | 1125 ± 21 | 345 ± 1 |
| LD-CuB + LD-MTX | 130 ± 4 | 1130 ± 14 | 355 ± 1 |
| HD-CuB + LD-MTX | 125 ± 7 | 990 ± 57 | 315 ± 1 |
| LD-CuB + VLD-MTX⁴ | 129 ± 5 | 1131 ± 23 | 332 ± 1 |

Note:
At the end of xenograft experiment (day 35), mice were sacrificed and organs were excised and weighed before formalin fixation.
Data represent mean weight ± standard deviation of five mice per group.
¹LD-CuB: Low-dose CuB (0.5 mg/kg body weight);
²HD-CuB: High-dose CuB (1.0 mg/kg body weight);
³LD-MTX: Low dose MTX (150 mg/kg body weight);
⁴VLD-MTX: very low dose methotrexate (50 mg/kg body weight).

TABLE 4
Effect of CuB and/or MTX on blood counts in mice

| Treatment | WBCs⁵ | Neutrophills | Lymphocytes | Monocytes | Eosinophils | Basophils | RBCs⁶ | Hb⁷ | Platelets |
|---|---|---|---|---|---|---|---|---|---|
| Group | (K/μl) | (K/μl) | (K/μl) | (K/μl) | (K/μl) | (K/μl) | (M/μl) | (g/dl) | (K/μl) |
| Normal Value | 1.8-10.7 | 0.1-2.4 | 0.9-9.3 | 0.0-0.4 | 0.0-0.2 | 0.0-0.2 | 6.36-9.42 | 11.0-15.1 | 592-2972 |
| Range | | | | | | | | | |
| Untreated | 5.25 ± 0.49 | 1.16 ± 0.49 | 3.86 ± 0.06 | 0.23 ± 0.01 | 0.02 ± 0.03 | 0.01 ± 0.01 | 9.16 ± 0.14 | 11.90 ± 0.14 | 726 ± 62.83 |
| LD-CuB¹ | 4.23 ± 0.04 | 1.42 ± 0.30 | 2.63 ± 0.24 | 0.18 ± 0.03 | 0.01 ± 0.01 | 0.00 ± 0.00 | 7.21 ± 1.00 | 9.60 ± 0.85 | 742 ± 115.97 |
| HD-CuB² | 5.55 ± 1.34 | 2.10 ± 0.42 | 2.90 ± 0.71 | 0.35 ± 0.07 | 0.15 ± 0.07 | 0.05 ± 0.07 | 7.78 ± 0.45 | 10.10 ± 0.42 | 651 ± 52.33 |
| LD-MTX³ | 4.80 ± 0.99 | 0.56 ± 0.42 | 4.05 ± 0.58 | 0.17 ± 0.04 | 0.00 ± 0.00 | 0.00 ± 0.00 | 7.69 ± 0.16 | 10.30 ± 0.42 | 690 ± 93 34 |
| LD-CuB + | 4.15 ± 0.78 | 1.57 ± 0.47 | 2.40 ± 0.28 | 0.20 ± 0.00 | 0.01 ± 0.01 | 0.00 ± 0.00 | 7.44 ± 0.08 | 10.35 ± 0.21 | 779 ± 131.52 |
| LD-MTX | | | | | | | | | |
| HD-CuB + | 4.55 ± 0.35 | 2.25 ± 0.21 | 2.10 ± 0.14 | 0.20 ± 0.00 | 0.03 ± 0.01 | 0.00 ± 0.00 | 7.91 ± 0.01 | 10.75 ± 0.21 | 808 ± 65.05 |
| LD-MTX | | | | | | | | | |
| LD-CuB + | 4.17 ± 0.63 | 1.46 ± 0.71 | 2.51 ± 0.23 | 0.18 ± 0.05 | 0.01 ± 0.01 | 0.00 ± 0.00 | 7.27 ± 0.56 | 10.11 ± 0.75 | 755 ± 97.00 |
| VLD-MTX⁴ | | | | | | | | | |

Note:
At the end of Xenograft experiment (day 35), whole blood samples were harvested by submandibular bleeding and complete blood counts (CBC) and differential cell analysis were done by the Hemagen Analyst Benchtop Chemistry System (Hemagen Diagnostics, Inc. Columbia, MD).
Data represent mean ± standard deviation of five mice per group.
¹LD-CuB: Low-dose CuB (0.5 mg/kg body weight);
²HD-CuB: High-dose CuB (1.0 mg/kg body weight);
³LD-MTX: Low-dose MTX (150 mg/kg body weight);
⁴VLD-MTX: very low-dose methotrexate (50 mg/kg body weight).
⁵WBC: white blood cells;
⁶RBC: red blood cells;
⁷Hb: hemoglobin.

TABLE 5
Effect of CuB and/or MTX on serum chemistry in mice

| Treatment | albumin | cholesterol | uric acid | CK¹⁰ | creatine | bilirubin | total protein | globulin |
|---|---|---|---|---|---|---|---|---|
| Group | (g/dl) | (mg/dl) | (mg/dl) | (U/l) | (mg/dl) | (mg/dl) | (g/dl) | (g/dl) |
| Untreated | 3.2 ± 0.0 | 143.0 ± 38.2 | 5.9 ± 0.8 | 115.0 ± 7.1 | 0.20 ± 0.01 | 0.06 ± 0.00 | 3.9 ± 0.2 | 0.7 ± 0.2 |
| LD-CuB¹ | 3.1 ± 0.1 | 147.0 ± 5.7 | 3.7 ± 0.3 | 140.5 ± 70.0 | 0.29 ± 0.05 | 0.09 ± 0.04 | 3.7 ± 1.0 | 0.7 ± 0.9 |
| HD-CuB² | 3.4 ± 0.1 | 172.0 ± 8.5 | 5.5 ± 0.6 | 156.0 ± 33.9 | 0.42 ± 0.25 | 0.06 ± 0.00 | 5.0 ± 0.1 | 1.6 ± 0.2 |
| LD-MTX³ | 3.3 ± 0.3 | 148.5 ± 24.7 | 4.7 ± 0.8 | 183.0 ± 140 | 0.39 ± 0.21 | 0.12 ± 0.00 | 4.6 ± 0.7 | 1.3 ± 0.4 |
| LD-CuB + | 2.7 ± 0.1 | 147.0 ± 43.8 | 5.2 ± 0.6 | 219.5 ± 179 | 0.26 ± 0.10 | 0.12 ± 0.00 | 3.9 ± 0.2 | 1.2 ± 0.1 |
| LD-MTX | | | | | | | | |
| HD-CuB + | 3.1 ± 0.2 | 141.0 ± 29.7 | 4.5 ± 0.1 | 81.0 ± 5.7 | 0.23 ± 0.05 | 0.06 ± 0 00 | 4.5 ± 0.1 | 1.4 ± 0.3 |
| LD-MTX | | | | | | | | |
| LD-CuB + | 3.0 ± 0.3 | 142.0 ± 33.1 | 5.4 ± 1.0 | 172.5 ± 71.8 | 0.29 ± 0.05 | 0.12 ± 0.04 | 3.8 ± 0.5 | 1.1 ± 0.7 |
| VLD-MTX⁴ | | | | | | | | |

Note:
At the end of Xenograft experiment (day 35), whole blood samples were harvested by submandibular bleeding and sera were separated using serum separator tubes. Serum chemistry was analyzed by the Hemagen Analyst Benchtop Chemistry System (Hemagen Diagnostics, Inc. Columbia, MD).
Data represent mean ± standard deviation of five mice per group.
¹LD-CuB: Low-dose CuB (0.5 mg/kg body weight);
²HD-CuB: High-dose CuB (1.0 mg/kg body weight);
³LD-MTX: Low-dose MTX (150 mg/kg body weight);
⁴VLD-MTX: very low-dose methotrexate (50 mg/kg body weight).
⁵ALP: alkaline phosphatase;
⁶GGT: gamma-glutamyl transpeptidase;
⁷glutamic-oxalacetic transaminase/aspartate aminotransferase;
⁸glutamic-pyruvic transaminas/alanine aminotransferase;
⁹BUN: blood urea nitrogen;
¹⁰CK: creatine kinase.

TABLE 6
Effect of CuB and/or MTX on clonogenic hematopoietic cells in mice

| Treatment group | CFU-GEMM⁵ | CFU-GM⁶ | BFU-E⁷ |
|---|---|---|---|
| Untreated | 4.9 ± 0.8 | 73.0 ± 2.7 | 14.9 ± 13.6 |
| LD-CuB¹ | 4.2 ± 0.8 | 69.8 ± 3.0 | 9.7 ± 1.4 |
| HD-CuB² | 3.8 ± 0.8 | 64.6 ± 4.6 | 8.3 ± 1.6 |
| LD-MTX³ | 3.4 ± 0.5 | 64.6 ± 3.6 | 7.1 ± 0.8 |
| LD-CuB + LD-MTX | 3.9 ± 0.8 | 64.9 ± 3.7 | 8.4 ± 1.1 |
| HD-CuB + LD-MTX | 3.9 ± 0.6 | 44.2 ± 6.1 | 6.1 ± 1.1 |
| LD-CuB + VLD-MTX⁴ | 4.0 ± 0.7 | 67.1 ± 3.9 | 8.3 ± 1.4 |

Note:
At the end of xenograft experiment (day 35), bone marrow mononuclear cells were harvested from murine femurs and cultured in 6-well plates at 2 × 10⁴ cells/well in MethoCult GF M3434 media optimized for colony-forming cell (CFC) assays.
Number of colonies was counted using light microscope after 2 weeks.
Data represent mean ± standard deviation of five mice per group.
¹LD-CuB: Low-dose CuB (0.5 mg/kg body weight);
²HD-CuB: High-dose CuB (1.0 mg/kg body weight);
³LD-MTX: Low-dose MTX (150 mg/kg body weight);
⁴VLD-MTX: very low-dose methotrexate (50 mg/kg body weight);
⁵CFU-GEMM: colony forming unit granulocyte/erythrocyte/monocyte/megakaryocyte;
⁶CFU-GM: colony-forming unit-granulocyte/macrophages;
⁷BFU-E: blast-forming unit erythroid.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

Many modifications and variations of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

1. Menon S, Manning B. Common corruption of the mTOR signaling network in human tumors. Oncogene. 2008; 27 Suppl 2:S43-51.
2. Guertin D, Sabatini D. Defining the role of mTOR in cancer. Cancer Cell. 2007; 12:9-22.
3. Petroulakis E, Mamane Y, Le Bacquer O, Shahbazian D, Sonenberg N. mTOR signaling: implications for cancer and anticancer therapy. Br J Cancer. 2006; 94:195-9.
4. Zhou Q, Deng Z, Zhu Y, Long H, Zhang S, Zhao J. mTOR/p70S6K Signal transduction pathway contributes to osteosarcoma progression and patients' prognosis. Med Oncol. 2009; DOI: 10.1007/s12032-009-9365-y.
5. Abdeen A, Chou A, Healey J, Khanna C, Osborne T, Hewitt S, et al. Correlation between clinical outcome and growth factor pathway expression in osteogenic sarcoma. Cancer. 2009; 115:5243-50.
6. Do S, Jung W, Kim H, Park Y. The expression of epidermal growth factor receptor and its downstream signaling molecules in osteosarcoma. Int J Oncol. 2009; 34:797-803.
7. Zhang W, Dziak R, Aletta J. EGF-mediated phosphorylation of extracellular signal-regulated kinases in osteoblastic cells. J Cell Physiol. 1995; 162:348-58.
8. Kiyokawa E, Takai S, Tanaka M, Iwase T, Suzuki M, Xiang Y, et al. Overexpression of ERK, an EPH family receptor protein tyrosine kinase, in various human tumors. Cancer Res. 1994; 54:3645-50.
9. Zhang Z, Neiva K, Lingen M, Ellis L, Nör J. VEGF-dependent tumor angiogenesis requires inverse and reciprocal regulation of VEGFR1 and VEGFR2. Cell Death Differ. 2010; 17:499-512.
10. Lee D, Iwanski G, Thoennissen N. Cucurbitacin: ancient compound shedding new light on cancer treatment. Scientific World Journal. 2010; 10:413-8.
11. Chen J, Chiu M, Nie R, Cordell G, Qiu S. Cucurbitacins and cucurbitane glycosides: structures and biological activities. Nat Prod Rep. 2005; 22:386-99.
12. Nishimura R, Moriyama K, Yasukawa K, Mundy G, Yoneda T. Combination of interleukin-6 and soluble interleukin-6 receptors induces differentiation and activation of JAK-STAT and MAP kinase pathways in MG-63 human osteoblastic cells. J Bone Miner Res. 1998; 13:777-85.
13. Bogenmann E, Moghadam H, DeClerck Y A, Mock A. c-myc Amplification and Expression in Newly Established Human Osteosarcoma Cell Lines. Cancer Res. 1987; 47(14):3808-14.
14. Lomenick B, Hao R, Jonai N, Chin R, Aghajan M, Warburton S, et al. Target identification using drug affinity responsive target stability (DARTS). Proc Natl Acad Sci USA. 2009; 106:21984-9.
15. Reagan-Shaw S, Nihal M, Ahmad N. Dose translation from animal to human studies revisited. FASEB J. 2008; 22:659-61.
16. Luong Q, O'Kelly J, Braunstein G, Hershman J, Koeffler H. Antitumor activity of suberoylanilide hydroxamic acid against thyroid cancer cell lines in vitro and in vivo. Clin Cancer Res. 2006; 12:5570-7.
17. Abramoff M D M, P. J. Ram, S. J. Image Processing with ImageJ. Biophotonics International. 2004; 11:7: 36-42.
18. Iwanski G, Lee D, En-Gal S, Doan N, Castor B, Vogt M, et al. Cucurbitacin B, a novel in vivo potentiator of gemcitabine with low toxicity in the treatment of pancreatic cancer. Br J Pharmacol. 2010; 160:998-1007.
19. Chou T, Talalay P. Quantitative analysis of dose-effect relationships: the combined effects of multiple drugs or enzyme inhibitors. Adv Enzyme Regul. 1984; 22:27-55.
20. Chou T. Drug combination studies and their synergy quantification using the Chou-Talalay method. Cancer Res. 2010; 70:440-6.
21. Pouysségur J, Dayan F, Mazure N. Hypoxia signalling in cancer and approaches to enforce tumour regression. Nature. 2006; 441:437-43.
22. Mariani B, Slate D, Schimke R. S phase-specific synthesis of dihydrofolate reductase in Chinese hamster ovary cells. Proc Natl Acad Sci USA. 1981; 78:4985-9.
23. Gorlick R, Goker E, Trippett T, Waltham M, Banerjee D, Bertino J. Intrinsic and acquired resistance to methotrexate in acute leukemia. N Engl J Med. 1996; 335:1041-8.
24. Lobo E, Balthasar J. Pharmacokinetic-pharmacodynamic modeling of methotrexate-induced toxicity in mice. J Pharm Sci. 2003; 92:1654-64.
25. Margolis S, Philips F, Sternberg S. The cytotoxicity of methotrexate in mouse small intestine in relation to inhibition of folic acid reductase and of DNA synthesis. Cancer Res. 1971; 31:2037-46.
26. Federman N, Bernthal N, Eilber F, Tap W. The multidisciplinary management of osteosarcoma. Curr Treat Options Oncol. 2009; 10:82-93.
27. Rosen G, Marcove R, Caparros B, Nirenberg A, Kosloff C, Huvos A. Primary osteogenic sarcoma: the rationale for preoperative chemotherapy and delayed surgery. Cancer. 1979; 43:2163-77.
28. Rosen G, Caparros B, Huvos A, Kosloff C, Nirenberg A, Cacavio A, et al. Preoperative chemotherapy for osteogenic sarcoma: selection of postoperative adjuvant chemotherapy based on the response of the primary tumor to preoperative chemotherapy. Cancer. 1982; 49:1221-30.
29. Guertin D, Sabatini D. The pharmacology of mTOR inhibition. Sci Signal. 2009; 2(67):pe24.
30. Sparks C, Guertin D. Targeting mTOR: prospects for mTOR complex 2 inhibitors in cancer therapy. Oncogene. 2010; 29(26):3733-44.
31. Rosner M, Siegel N, Valli A, Fuchs C, Hengstschläger M. mTOR phosphorylated at S2448 binds to raptor and rictor. Amino Acids. 2010; 38:223-8.
32. Feldman M, Apsel B, Uotila A, Loewith R, Knight Z, Ruggero D, et al. Active-site inhibitors of mTOR target rapamycin-resistant outputs of mTORC1 and mTORC2. PLoS Biol. 2009; 7:e38.
33. Diddens H, Niethammer D, Jackson R. Patterns of cross-resistance to the antifolate drugs trimetrexate, metoprine, homofolate, and CB3717 in human lymphoma and osteosarcoma cells resistant to methotrexate. Cancer Res. 1983; 43:5286-92.
34. Teachey D, Sheen C, Hall J, Ryan T, Brown V, Fish J, et al. mTOR inhibitors are synergistic with methotrexate: an effective combination to treat acute lymphoblastic leukemia. Blood. 2008; 112:2020-3.
35. Müller H, Lukas J, Schneider A, Warthoe P, Bartek J, Eilers M, et al. Cyclin D1 expression is regulated by the retinoblastoma protein. Proc Natl Acad Sci USA. 1994; 91:2945-9.
36. Serra M, Reverter-Branchat G, Maurici D, Benini S, Shen J, Chano T, et al. Analysis of dihydrofolate reductase and reduced folate carrier gene status in relation to methotrexate resistance in osteosarcoma cells. Ann Oncol. 2004; 15:151-60.
37. Bruheim S, Bruland O, Breistol K, Maelandsmo G, Fodstad O. Human osteosarcoma xenografts and their sensitivity to chemotherapy. Pathol Oncol Res. 2004; 10:133-41.
38. Gazitt Y, Kolaparthi V, Moncada K, Thomas C, Freeman J. Targeted therapy of human osteosarcoma with 17AAG or rapamycin: characterization of induced apoptosis and inhibition of mTOR and Akt/MAPK/Wnt pathways. Int J Oncol. 2009; 34:551-61.

What is claimed is:

1. A method of treating cancer in a mammal, comprising:
administering a quantity of cucurbitacin B (CuB) with a quantity of methotrexate (MTX) to the mammal in need thereof, in an amount effective to treat the cancer,
wherein CuB and/or MTX is a pharmaceutical equivalent, analog, derivative, salt or prodrug thereof.

2. The method according to claim 1, wherein the cancer is osteosarcoma.

3. The method according to claim 1, wherein the administration of the combination of CuB and MTX has a synergistic effect in the treatment of cancer.

4. The method according to claim 1, wherein the quantity of CuB is administered in a low dose.

5. The method according to claim 4, wherein the low dose quantity of CuB is administered every one to three days.

6. The method according to claim 1, wherein the quantity of MTX is administered in a low dose.

7. The method according to claim 6, wherein the low dose quantity of MTX is administered every one to three weeks.

8. A method of inducing apoptosis in cancer cells in a mammal, comprising:
administering a quantity of CuB with a quantity of MTX to the mammal in need thereof, in an amount effective to induce apoptosis of the cancer cells in the mammal,
wherein CuB and/or MTX is a pharmaceutical equivalent, analog, derivative, salt or prodrug thereof.

9. The method according to claim 8, wherein the cancer is osteosarcoma.

10. The method according to claim 8, wherein the administration of the combination of CuB and MTX has a synergistic effect in the treatment of cancer.

11. The method according to claim 8, wherein the quantity of CuB is administered in a low dose.

12. The method according to claim 11, wherein the low dose quantity of CuB is administered every one to three days.

13. The method according to claim 8, wherein the quantity of MTX is administered in a low dose.

14. The method according to claim 13, wherein the low dose quantity of MTX is administered every one to three weeks.

15. A method of preventing metastases of cancer in a mammal, comprising:
administering a quantity of CuB with a quantity of MTX to the mammal in need thereof, in an amount effective to prevent metastases of the cancer in the mammal,
wherein CuB and/or MTX is a pharmaceutical equivalent, analog, derivative, salt or prodrug thereof.

16. The method according to claim 16, wherein the cancer is osteosarcoma.

17. The method according to claim 17, wherein the administration of the combination of CuB and MTX has a synergistic effect in the treatment of cancer.

18. The method according to claim 17, wherein the quantity of CuB is administered in a low dose.

19. The method according to claim 18, wherein the low dose quantity of CuB is administered every one to three days.

20. The method according to claim 16, wherein the quantity of MTX is administered in a low dose.

21. The method according to claim 20, wherein the low dose quantity of MTX is administered every one to three weeks.

22. A method of reducing the likelihood metastases of cancer in a mammal, comprising:
administering a quantity of CuB with a quantity of MTX to the mammal in need thereof, in an amount effective to prevent metastases of the cancer in the mammal,
wherein CuB and/or MTX is a pharmaceutical equivalent, analog, derivative, salt or prodrug thereof.

23. The method according to claim 22, wherein the cancer is osteosarcoma.

24. The method according to claim 22, wherein the administration of the combination of CuB and MTX has a synergistic effect in the treatment of cancer.

25. The method according to claim 22, wherein the quantity of CuB is administered in a low dose.

26. The method according to claim 25, wherein the low dose quantity of CuB is administered every one to three days.

27. The method according to claim 22, wherein the quantity of MTX is administered in a low dose.

28. The method according to claim 27, wherein the low dose quantity of MTX is administered every one to three weeks.

29. A composition for treating cancer in a mammal, comprising:
a quantity of CuB and a quantity of MTX,
wherein CuB and/or MTX is pharmaceutical equivalent, analog, derivative, salt or prodrug thereof.

30. A composition for inducing apoptosis of cancer cells in a mammal, comprising:
a quantity of CuB and a quantity of MTX,
wherein CuB and/or MTX is pharmaceutical equivalent, analog, derivative, salt or prodrug thereof.

31. A composition for preventing metastases or reducing the likelihood of metastases of cancer in a mammal, comprising:
a quantity of CuB and a quantity of MTX,
wherein CuB and/or MTX is pharmaceutical equivalent, analog, derivative, salt or prodrug thereof.

32. A kit for treating cancer comprising a composition comprising:
a quantity of CuB, a quantity of MTX, and
instructions for use,
wherein CuB and/or MTX is pharmaceutical equivalent, analog, derivative, salt or prodrug thereof.
